(19) United States
(12) Patent Application Publication
(10) Pub. No.: US 2008/0188562 A1
(43) Pub. Date: Aug. 7, 2008

(54) TREATING TINNITUS USING PRODRUGS OF GABAPENTIN AND PREGABALIN

(76) Inventor: Noa Zerangue, Portola Valley, CA (US)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/939,517

(22) Filed: Nov. 13, 2007

Related U.S. Application Data
(60) Provisional application No. 60/859,283, filed on Nov. 14, 2006.

(51) Int. Cl. A61K 31/195; A61P 27/16

(52) U.S. Cl. 514/561

(57) ABSTRACT

Methods of using prodrugs of gabapentin or pregabalin and pharmaceutical compositions thereof to treat tinnitus, and pharmaceutical compositions of prodrugs of gabapentin or pregabalin useful in treating tinnitus are disclosed.

This application claims the benefit under 35. U.S.C. § 119 of U.S. Provisional Application No. 60/859,283, filed Nov. 14, 2006, which is incorporated by reference herein in its entirety.

### FIELD

Methods and compositions disclosed herein relate to methods of using prodrugs of gabapentin and pregabalin and pharmaceutical compositions thereof to treat tinnitus in patients and to pharmaceutical compositions of prodrugs of gabapentin and pregabalin useful in treating tinnitus.

### BACKGROUND

Tinnitus is the perception of sound in the absence of acoustic stimulation, and often involves sound sensations such as ringing, buzzing, roaring, whistling, and hissing that cannot be attributed to an external sound source. Tinnitus is a symptom associated with many forms of hearing loss and can also be a symptom of other health problems. It is estimated that 40 million people in the United States experience chronic tinnitus and 10 million of these people consider their tinnitus to be serious problem (see e.g., Vio and Holm, Drug Discovery Today 2005, 10, 1263-1265; Cooper, J Am Acad Audiol 1994, 5, 37-43; and Henry et al., J Rehabil Res Dev 2003, 40, 157-177).

Tinnitus can be caused by hearing loss, loud noise, medicine, and other health problems such as allergies, head and neck tumors, cardiovascular disorders such as atherosclerosis, high blood pressure, turbulent blood flow, malformation of capillaries, trauma such as excessive exposure to loud noise, long-term use of certain medications such as salicylates, quinine, cisplatin and certain types of antibiotics, changes to ear bones such as otosclerosis, and jaw and neck injuries. In general, insults or damage to the auditory and somatosensory systems can create an imbalance between inhibitory and excitatory transmitter actions in the midbrain, auditory cortex and brain stem (see e.g., Eggermont, Drug Discovery Today 2005, 10(19), 1283-1290). This imbalance can cause hyperexcitability of auditory neurons that can lead to the perception of phantom sounds. For acute tinnitus such as induced by drugs or loud noises, increased spontaneous firing rates in the auditory nerve fibers have been attributed to reduced levels of central inhibition, presumably by GABA, in central auditory structures leading to neural hyperactivity in the inferior colliculus (Bauer et al., Hear. Res. 2000, 147, 175-82; Abbott et al., Neuroscience 1999, 93, 1375-81; Milbrandt, et al., Hear. Res. 2000, 147, 251-60; and Salvi et al., Hear Res 2000, 147, 261-74). Although chronic tinnitus may have a different cause than acute tinnitus, reduced GABA levels have also been implicated (Caspary et al., Neuroscience 1999, 93, 307-312).

The γ-aminobutyric acid (γ-aminobutyric acid is abbreviated herein as GABA) analog, gabapentin (1), has been approved in the United States for the treatment of epileptic seizures, diabetic neuropathy, post-herpetic neuralgia, and restless legs syndrome (Backonja et al., JAMA 1998, 280, 1831-36; and Rose et al., Anaesthesia 2002, 57, 451-62). Gabapentin has also shown efficacy in controlled studies for treating neuropathic pain of various etiologies. Consistent with the hypothesis that loss of GABA inhibition of the central auditory pathway may cause or contribute to tinnitus, gabapentin has been shown effective in reversibly attenuating acoustic trauma-induced tinnitus in animals (Ibauer and Brozoski, J Assoc Res Otolarynology, 2001, 2(1), 54-64). Studies also suggest that gabapentin can be useful in treating tinnitus in humans (see e.g., Bauer and Brozoski, Laryngoscope 2006, 116, 675-681; Zapp, Ear Nose Throat J 2001, 80, 114-116; and Shulman et al., Int Tinnitus J 2002, 8, 30-33; but see Witsell et al., Otology & Neurotology 2006, 28, 11-15; and Piccirillo et al., Arch Otolaryngol Head Neck Surg. 2007, 133(4), 390-7). Methods of using derivatives of GABA for the treatment of tinnitus have also been disclosed in Dooley and Wustrow, U.S. Pat. No. 7,026,505; Dooley and Wustrow, U.S. Application Publication No. 2006/0100281; and Donevan et al., U.S. Application Publication No. 2005/0070483. The broad pharmaceutical activities of GABA analogs such as gabapentin (1) and pregabalin (2):

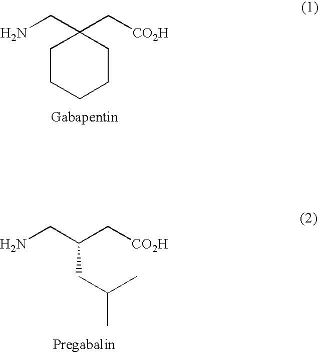

has stimulated intensive interest in preparing related compounds that have superior pharmaceutical properties in comparison to GABA, e.g., the ability to cross the blood-brain barrier (see, e.g., Satzinger et al., U.S. Pat. No. 4,024,175; Silverman et al., U.S. Pat. No. 5,563,175; Horwell et al., U.S. Pat. No. 6,020,370; Silverman et al., U.S. Pat. No. 6,028,214; Horwell et al., U.S. Pat. No. 6,103,932; Silverman et al., U.S. Pat. No. 6,117,906; Silverman, International Publication No. WO92/09560; Silverman et al., International Publication No. WO 93/23383; Horwell et al., International Publication No. WO 97/29101, Horwell et al., International Publication No. WO 97/33858; Horwell et al., International Publication No. WO 97/33859; Bryans et al., International Publication No. WO 98/17627; Guglietta et al., International Publication No. WO 99/08671; Bryans et al., International Publication No. WO 99/21824; Bryans et al., International Publication No. WO 99/31057; Belliotti et al., International Publication No. WO 99/31074; Bryans et al., International Publication No. WO 99/31075; Bryans et al., International Publication No. WO 99/61424; Bryans et al., International Publication No. WO 00/15611; Belliot et al., International Publication No. WO 00/31020; Bryans et al., International Publication No. WO 00/50027; and Bryans et al., International Publication No. WO 02/00209).

One significant problem associated with the clinical use of many GABA analogs, including gabapentin and pregabalin, is rapid systemic clearance. Consequently, these drugs require frequent dosing to maintain a therapeutic or prophylactic concentration in the systemic circulation (Bryans et al., Med. Res. Rev. 1999, 19, 149-177). For example, dosing regimens of 300-600 mg doses of gabapentin administered three times per day are typically used for anticonvulsive therapy. Higher doses (1,800-3,600 mg/day in three or four divided doses) are typically used for the treatment of neuropathic pain states. Doses of gabapentin up to 2,400 mg/day have been shown to be effective in treating tinnitus (Bauer and Brozoski, Laryngoscope 2006, 116, 675-681).

Although oral sustained released formulations are conventionally used to reduce the dosing frequency of drugs that exhibit rapid systemic clearance, oral sustained release formulations of gabapentin and pregabalin have not been developed because these drugs are not absorbed via the large intestine. Rather, these compounds are typically absorbed in the small intestine by one or more amino acid transporters (e.g., the “large neutral amino acid transporter,” see Jezyk et al., Pharm. Res. 1999, 16, 519-526). The limited residence time of both conventional and sustained release oral dosage forms in the proximal absorptive region of the gastrointestinal tract necessitates frequent daily dosing of conventional oral dosage forms of these drugs, and has prevented the successful application of sustained release technologies to many GABA analogs.

One method for overcoming rapid systemic clearance of GABA analogs is to administer an extended release dosage formulation containing a colonically absorbed GABA analog prodrug (Gallop et al., U.S. Pat. Nos. 6,818,787, 6,972,341, and 7,026,351; and International Publication Nos. WO 2002/100347 and WO 2002/100349, each of which is incorporated by reference herein in its entirety). Sustained release formulations enable a colonically absorbed GABA analog prodrug to be absorbed over a wider region of the gastrointestinal tract than the parent drug including across the wall of the colon where sustained release oral dosage forms typically spend a significant portion of gastrointestinal transit time. These prodrugs are typically converted to the parent GABA analog upon absorption in vivo.

### SUMMARY

Currently, there is no FDA approved drug for treating tinnitus. Furthermore, therapeutic agents for treating tinnitus either have significant side effects or are rapidly systemically cleared. Therefore, there is a need in the art for a method of treating tinnitus by delivering an agent, such as a prodrug of gabapentin or pregabalin, particularly for example, in an extended release dosage form, with a reduced rate of systemic clearance, and without significant side effects.

In a first aspect, methods of treating tinnitus in a patient are provided comprising administering to a patient in need of such treatment a therapeutically effective amount of at least one compound chosen from Formula (I) and Formula (II):

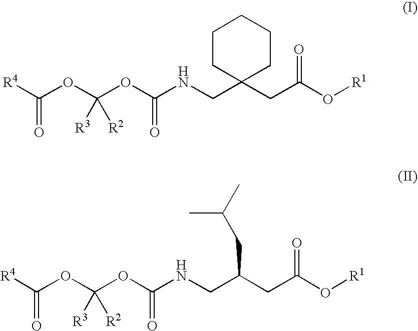

or a pharmaceutically acceptable salt thereof, a pharmaceutically acceptable solvate of any of the foregoing, and a pharmaceutically acceptable N-oxide of any of the foregoing, wherein:

R¹ is chosen from hydrogen, alkyl, substituted alkyl, aryl, substituted aryl, arylalkyl, substituted arylalkyl, cycloalkyl, substituted cycloalkyl, cycloheteroalkyl, substituted cycloheteroalkyl, heteroalkyl, substituted heteroalkyl, heteroaryl, substituted heteroaryl, heteroarylalkyl, and substituted heteroarylalkyl;

R² and R³ are each independently chosen from hydrogen, alkyl, substituted alkyl, alkoxycarbonyl, substituted alkoxycarbonyl, aryl, substituted aryl, arylalkyl, substituted arylalkyl, carbamoyl, substituted carbamoyl, cycloalkyl, substituted cycloalkyl, heteroalkyl, substituted heteroalkyl, cycloheteroalkyl, substituted cycloheteroalkyl, heteroaryl, substituted heteroaryl, heteroarylalkyl, and substituted heteroarylalkyl, or R² and R³ together with the carbon atom to which they are bonded form a cycloalkyl, substituted cycloalkyl, cycloheteroalkyl, or substituted cycloheteroalkyl ring; and

R⁴ is chosen from acyl, substituted acyl, alkyl, substituted alkyl, aryl, substituted aryl, arylalkyl, substituted arylalkyl, cycloalkyl, substituted cycloalkyl, cycloheteroalkyl, substituted cycloheteroalkyl, heteroalkyl, substituted heteroalkyl, heteroaryl, substituted heteroaryl, heteroarylalkyl, and substituted heteroarylalkyl.

In a second aspect, methods of treating tinnitus in a patient are provided comprising administering to a patient in need of such treatment a pharmaceutical composition comprising a therapeutically effective amount of at least one compound chosen from Formula (I) and Formula (II), a pharmaceutically acceptable salt thereof, a pharmaceutically acceptable solvate of any of the foregoing, and a pharmaceutically acceptable N-oxide of any of the foregoing, and a pharmaceutically acceptable vehicle.

### DETAILED DESCRIPTION

### Definitions

A dash (“-”) that is not between two letters or symbols is used to indicate a point of attachment for a moiety or substituent. For example, the moiety CONH₂ is attached through the carbon atom.

“Alkyl” by itself or as part of another substituent refers to a saturated or unsaturated, branched or straight-chain, monovalent hydrocarbon radical derived by the removal of one hydrogen atom from a single carbon atom of a parent alkane, alkene, or alkyne. Examples of alkyl groups include, but are not limited to, methyl; ethyls such as ethanyl, ethenyl, and ethynyl; propyls such as propan-1-yl, propan-2-yl, prop-1-en-1-yl, prop-1-en-2-yl, prop-2-en-1-yl(allyl), prop-1-yn-1-yl, prop-2-yn-1-yl, etc.; butyls such as butan-1-yl, butan-2-yl, 2-methyl-propan-1-yl, 2-methyl-propan-2-yl, but-1-en-1-yl, but-1-en-2-yl, 2-methyl-prop-1-en-1-yl, but-2-en-1-yl, but-2-en-2-yl, buta-1,3-dien-1-yl, buta-1,3-dien-2-yl, but-1-yn-1-yl, but-1-yn-3-yl, but-3-yn-1-yl, etc.; and the like.

The term “alkyl” is specifically intended to include groups having any degree or level of saturation, i.e., groups having exclusively single carbon-carbon bonds, groups having one or more double carbon-carbon bonds, groups having one or more triple carbon-carbon bonds, and groups having mixtures of single, double, and triple carbon-carbon bonds. Where a specific level of saturation is intended, the terms “alkanyl,” “alkenyl,” and “alkynyl” are used. In certain embodiments, an alkyl group may comprise from 1 to 20 carbon atoms, in certain embodiments, from 1 to 10 carbon atoms, in certain embodiments, from 1 to 6 carbon atoms, and in certain embodiments, from 1 to 3 carbon atoms.

“Alkanyl” by itself or as part of another substituent refers to a saturated branched or straight-chain alkyl radical derived by the removal of one hydrogen atom from a single carbon atom of a parent alkane. Examples of alkanyl groups include, but are not limited to, methanyl; ethanyl; propanyls such as propan-1-yl and propan-2-yl (isopropyl), etc.; butanyls such as butan-1-yl, butan-2-yl(sec-butyl), 2-methyl-propan-1-yl(isobutyl), 2-methyl-propan-2-yl(t-butyl), etc.; and the like.

“Alkenyl” by itself or as part of another substituent refers to an unsaturated branched or straight-chain alkyl radical having at least one carbon-carbon double bond derived by the removal of one hydrogen atom from a single carbon atom of a parent alkene. The group may be in either the cis or trans conformation about the double bond(s). Examples of alkenyl groups include, but are not limited to, ethenyl; propenyls such as prop-1-en-1-yl, prop-1-en-2-yl, prop-2-en-1-yl(allyl), and prop-2-en-2-yl; butenyls such as but-1-en-1-yl, but-1-en-2-yl, 2-methyl-prop-1-en-1-yl, but-2-en-1-yl, but-2-en-1-yl, but-2-en-2-yl, buta-1,3-dien-1-yl, buta-1,3-dien-2-yl, etc.; and the like.

“Alkynyl” by itself or as part of another substituent refers to an unsaturated branched or straight-chain alkyl radical having at least one carbon-carbon triple bond derived by the removal of one hydrogen atom from a single carbon atom of a parent alkyne. Examples of alkynyl groups include, but are not limited to, ethynyl; propynyls such as prop-1-yn-1-yl, prop-2-yn-1-yl, etc.; butynyls such as but-1-yn-1-yl, but-1-yn-3-yl, but-3-yn-1-yl, etc.; and the like.

“Acyl” by itself or as part of another substituent refers to a radical —C(O)R³⁰, where R³⁰ is chosen from hydrogen, alkyl, cycloalkyl, cycloheteroalkyl, aryl, arylalkyl, heteroalkyl, heteroaryl, and heteroarylalkyl, as defined herein. Examples of acyl groups include, but are not limited to, formyl, acetyl, cyclohexylcarbonyl, cyclohexylmethylcarbonyl, benzoyl, benzylcarbonyl, and the like.

“Alkoxy” by itself or as part of another substituent refers to a radical —OR³¹ where R³¹ is chosen from alkyl, cycloalkyl, cycloalkylalkyl, aryl, and arylalkyl, as defined herein. Examples of alkoxy groups include, but are not limited to, methoxy, ethoxy, propoxy, butoxy, cyclohexyloxy, and the like.

“Alkoxycarbonyl” by itself or as part of another substituent refers to a radical —C(O)OR³² where R³² is alkyl, as defined herein. Examples of alkoxycarbonyl groups include, but are not limited to, methoxycarbonyl, ethoxycarbonyl, propoxycarbonyl, and butoxycarbonyl, and the like.

“Aryl” by itself or as part of another substituent refers to a monovalent aromatic hydrocarbon radical derived by the removal of one hydrogen atom from a single carbon atom of a parent aromatic ring system. Aryl encompasses 5- and 6-membered carbocyclic aromatic rings, for example, benzene; bicyclic ring systems wherein at least one ring is carbocyclic and aromatic, for example, naphthalene, indane, and tetralin; and tricyclic ring systems wherein at least one ring is carbocyclic and aromatic, for example, fluorene. Aryl encompasses multiple ring systems having at least one carbocyclic aromatic ring fused to at least one carbocyclic aromatic ring, cycloalkyl ring, or heterocycloalkyl ring. For example, aryl includes 5- and 6-membered carbocyclic aromatic rings fused to a 5- to 7-membered heterocycloalkyl ring containing one or more heteroatoms chosen from N, O, and S. For such fused, bicyclic ring systems wherein only one of the rings is a carbocyclic aromatic ring, the point of attachment may be at the carbocyclic aromatic ring or the heterocycloalkyl ring. Examples of aryl groups include, but are not limited to, groups derived from aceanthrylene, acenaphthylene, acephenanthrylene, anthracene, azulene, benzene, chrysene, coronene, fluoranthene, fluorene, hexacene, hexaphene, hexylene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, trinaphthalene, and the like. In certain embodiments, an aryl group may comprise from 5 to 20 carbon atoms, and in certain embodiments, from 5 to 12 carbon atoms. Aryl, however, does not encompass or overlap in any way with heteroaryl, separately defined herein.

“Arylalkyl” by itself or as part of another substituent refers to an acyclic alkyl radical in which one of the hydrogen atoms bonded to a carbon atom, typically a terminal or sp³ carbon atom, is replaced with an aryl group. Examples of arylalkyl groups include, but are not limited to, benzyl, 2-phenylethan-1-yl, 2-phenylethen-1-yl, naphthylmethyl, 2-naphthylethan-1-yl, 2-naphthylethen-1-yl, naphthobenzyl, 2-naphthophenylethan-1-yl, and the like. Where specific alkyl moieties are intended, the nomenclature arylalkanyl, arylalkenyl, or arylalkynyl is used. In certain embodiments, an arylalkyl group is C₇₋₃₀ arylalkyl, e.g., the alkanyl, alkenyl, or alkynyl moiety of the arylalkyl group is C₁₋₁₀ and the aryl moiety is C₆₋₂₀, and in certain embodiments, an arylalkyl group is C₇₋₂₀ arylalkyl, e.g., the alkanyl, alkenyl, or alkynyl moiety of the arylalkyl group is C₁₋₈ and the aryl moiety is C₆₋₁₂.

“AUC” is the area under a curve representing the concentration of a compound or metabolite thereof in a biological fluid in a patient as a function of time following administration of the compound to the patient. In certain embodiments, the compound can be a prodrug and the metabolite can be a drug. Examples of biological fluids include plasma and blood. The AUC may be determined by measuring the concentration of a compound or metabolite thereof in a biological fluid such as the plasma or blood using methods such as liquid chromatography-tandem mass spectrometry (LC/MS/MS), at various time intervals, and calculating the area under the plasma concentration-versus-time curve. Suitable methods for calculating the AUC from a drug concentration-versus-time curve are well known in the art. For example, an AUC for gabapentin or pregabalin may be determined by measuring the concentration of gabapentin or pregabalin in the plasma or blood of a patient following administration of a compound of Formula (I) or Formula (II) to the patient.

“Carbamoyl” by itself or as part of another substituent refers to the radical —C(O)NR⁴⁰R⁴¹ where R⁴⁰ and R⁴¹ are independently chosen from hydrogen, alkyl, cycloalkyl, and aryl as defined herein.

“Cₘₐₓ,” is the maximum concentration of a drug in the plasma or blood of a patient following administration of a dose of the drug or prodrug to the patient.

“Tₘₐₓ” is the time to the maximum concentration (Cₘₐₓ) of a drug in the plasma or blood of a patient following administration of a dose of the drug or prodrug to the patient

“Compounds” of Formula (I) and Formula (II) disclosed herein include any specific compounds encompassed by the corresponding structures. Compounds may be identified either by their chemical structure and/or chemical name. When the chemical structure and chemical name conflict, the chemical structure is determinative of the identity of the compound. The compounds described herein may contain one or more chiral centers and/or double bonds and therefore may exist as stereoisomers such as double-bond isomers (i.e., geometric isomers), enantiomers, or diastereomers. Accordingly, any chemical structures within the scope of the specification depicted, in whole or in part, with a relative configuration encompass all possible enantiomers and stereoisomers of the illustrated compounds including the stereoisomerically pure form (e.g., geometrically pure, enantiomerically pure, or diastereomerically pure) and enantiomeric and stereoisomeric mixtures. Enantiomeric and stereoisomeric mixtures may be resolved into their component enantiomers or stereoisomers using separation techniques or chiral synthesis techniques well known to the skilled artisan.

Compounds of Formula (I) and Formula (II) include, but are not limited to, optical isomers of compounds of Formula (I) and Formula (II), racemates thereof, and other mixtures thereof. In such embodiments, the single enantiomers or diastereomers, i.e., optically active forms, may be obtained by asymmetric synthesis or by resolution of the racemates. Resolution of the racemates may be accomplished, for example, by conventional methods such as crystallization in the presence of a resolving agent, or chromatography, using, for example a chiral high-pressure liquid chromatography (HPLC) column. In addition, compounds of Formula (I) and Formula (II) include Z- and E-forms (or cis- and trans-forms) of compounds with double bonds. In embodiments in which compounds of Formula (I) and Formula (II) exist in various tautomeric forms, compounds provided by the present disclosure include all tautomeric forms of the compounds.

The compounds of Formula (I) and Formula (II) may also exist in several tautomeric forms including the enol form, the keto form, and mixtures thereof. Accordingly, the chemical structures depicted herein encompass all possible tautomeric forms of the illustrated compounds. The compounds of Formula (I) and Formula (II) also include isotopically labeled compounds where one or more atoms have an atomic mass different from the atomic mass conventionally found in nature. Examples of isotopes that may be incorporated into the compounds disclosed herein include, but are not limited to, ²H, ³H, ¹¹C, ¹³C, ¹⁴C, ¹⁵N, ¹⁸O, ¹⁷O, etc. Compounds may exist in unsolvated forms as well as solvated forms, including hydrated forms, and as N-oxides. In general, compounds may be hydrated, solvated, or N-oxides. Certain compounds may exist in multiple crystalline or amorphous forms. Compounds of Formula (I) and Formula (II) include pharmaceutically acceptable salts thereof, or pharmaceutically acceptable solvates of the free acid form of any of the foregoing, as well as crystalline forms of any of the foregoing.

“Corresponding GABA analog” means gabapentin when the prodrug has the structure of Formula (I) and pregabalin when the prodrug has the structure of Formula (II).

“Cycloalkyl” by itself or as part of another substituent refers to a saturated or partially unsaturated cyclic alkyl radical. Where a specific level of saturation is intended, the nomenclature “cycloalkanyl” or “cycloalkenyl” is used. Examples of cycloalkyl groups include, but are not limited to, groups derived from cyclopropane, cyclobutane, cyclopentane, cyclohexane, and the like. In certain embodiments, a cycloalkyl group is C₃₋₁₅ cycloalkyl, and in certain embodiments, C₅₋₁₂ cycloalkyl.

“Cycloheteroalkyl” by itself or as part of another substituent refers to a saturated or partially unsaturated cyclic alkyl radical in which one or more carbon atoms (and any associated hydrogen atoms) are independently replaced with the same or different heteroatom. Typical heteroatoms to replace the carbon atom(s) include, but are not limited to, N, P, O, S, Si, etc. Where a specific level of saturation is intended, the nomenclature “cycloheteroalkanyl” or “cycloheteroalkenyl” is used. Examples of cycloheteroalkyl groups include, but are not limited to, groups derived from epoxides, azirines, thiiranes, imidazolidine, morpholine, piperazine, piperidine, pyrazolidine, pyrrolidine, quinuclidine, and the like.

“GABA analog” refers to gabapentin (1) or pregabalin (2).

“Halogen” refers to a fluoro, chloro, bromo, or iodo group.

“Heteroalkyl” by itself or as part of another substituent refer to an alkyl group in which one or more of the carbon atoms (and any associated hydrogen atoms) are independently replaced with the same or different heteroatomic group(s). Examples of heteroatomic groups include, but are not limited to, —O—, —S—, —O—O—, —S—S—, —O—S—, —NR³⁷—, ═N—N═, —N═N—, —N═N—NR⁵⁰R⁵¹, —PR⁵²—, —P(O)₂—, —POR⁴²—, —O—P(O)₂—, —SO—, —SO₂—, —SnR⁴³R⁴⁴—, and the like, where R³⁷, R42, R43, R⁴⁴, R⁵⁰, R⁵¹, and R⁵² are independently chosen from hydrogen, alkyl, substituted alkyl, aryl, substituted aryl, arylalkyl, substituted arylalkyl, cycloalkyl, substituted cycloalkyl, cycloheteroalkyl, substituted cycloheteroalkyl, heteroalkyl, substituted heteroalkyl, heteroaryl, substituted heteroaryl, heteroarylalkyl, and substituted heteroarylalkyl. Where a specific level of saturation is intended, the nomenclature “heteroalkanyl,” “heteroalkenyl,” or “heteroalkynyl” is used.

“Heteroaryl” by itself or as part of another substituent refers to a monovalent heteroaromatic radical derived by the removal of one hydrogen atom from a single atom of a parent heteroaromatic ring system. Heteroaryl encompasses multiple ring systems having at least one heteroaromatic ring fused to at least one other ring, which may be aromatic or non-aromatic. Heteroaryl encompasses 5- to 7-membered aromatic, monocyclic rings containing one or more, for example, from 1 to 4, or in certain embodiments, from 1 to 3, heteroatoms chosen from N, O, and S, with the remaining ring atoms being carbon; and bicyclic heterocycloalkyl rings containing one or more, for example, from 1 to 4, or in certain embodiments, from 1 to 3, heteroatoms chosen from N, O, and S, with the remaining ring atoms being carbon and wherein at least one heteroatom is present in an aromatic ring. For example, heteroaryl includes a 5- to 7-membered heteroaromatic ring fused to a 5- to 7-membered cycloalkyl ring. For such fused, bicyclic heteroaryl ring systems wherein only one of the rings contains one or more heteroatoms, the point of attachment may be at the heteroaromatic ring or the cycloalkyl ring. In certain embodiments, when the total number of N, S, and O atoms in the heteroaryl group exceeds one, the heteroatoms are not adjacent to one another. In certain embodiments, the total number of N, S, and O atoms in the heteroaryl group is not more than two. In certain embodiments, the total number of N, S, and O atoms in the aromatic heterocycle is not more than one. Heteroaryl does not encompass or overlap with aryl as defined herein.

Examples of heteroaryl groups include, but are not limited to, groups derived from acridine, arsindole, carbazole, β-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene, and the like. In certain embodiments, a heteroaryl group is from 5- to 20-membered heteroaryl, and in certain embodiments from 5- to 10-membered heteroaryl. In certain embodiments heteroaryl groups are those derived from thiophene, pyrrole, benzothiophene, benzofuran, indole, pyridine, quinoline, imidazole, oxazole, or pyrazine.

“Heteroarylalkyl” by itself or as part of another substituent refers to an acyclic alkyl radical in which one of the hydrogen atoms bonded to a carbon atom, typically a terminal or sp³ carbon atom, is replaced with a heteroaryl group. Where specific alkyl moieties are intended, the nomenclature “heteroarylalkanyl,” “heteroarylalkenyl,” or “heterorylalkynyl” is used. In certain embodiments, a heteroarylalkyl group is a 6- to 30-membered heteroarylalkyl, e.g., the alkanyl, alkenyl, or alkynyl moiety of the heteroarylalkyl is 1- to 10-membered and the heteroaryl moiety is a 5- to 20-membered heteroaryl, and in certain embodiments, 6- to 20-membered heteroarylalkyl, e.g., the alkanyl, alkenyl, or alkynyl moiety of the heteroarylalkyl is 1- to 8-membered and the heteroaryl moiety is a 5- to 12-membered heteroaryl.

“N-oxide” refers to the zwitterionic nitrogen oxide of a tertiary amine base.

“Parent aromatic ring system” refers to an unsaturated cyclic or polycyclic ring system having a conjugated π electron system. Included within the definition of “parent aromatic ring system” are fused ring systems in which one or more of the rings are aromatic and one or more of the rings are saturated or unsaturated, such as, for example, fluorene, indane, indene, phenalene, etc. Examples of parent aromatic ring systems include, but are not limited to, aceanthrylene, acenaphthylene, acephenanthrylene, anthracene, azulene, benzene, chrysene, coronene, fluoranthene, fluorene, hexacene, hexaphene, hexylene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, trinaphthalene, and the like.

“Parent heteroaromatic ring system” refers to a parent aromatic ring system in which one or more carbon atoms (and any associated hydrogen atoms) are independently replaced with the same or different heteroatom. Examples of heteroatoms to replace the carbon atoms include, but are not limited to, N, P, O, S, and Si, etc. Specifically included within the definition of “parent heteroaromatic ring systems” are fused ring systems in which one or more of the rings are aromatic and one or more of the rings are saturated or unsaturated, such as, for example, arsindole, benzodioxan, benzofuran, chromane, chromene, indole, indoline, xanthene, etc. Examples of parent heteroaromatic ring systems include, but are not limited to, arsindole, carbazole, β-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene, and the like.

“Patient” refers to a mammal, for example, a human.

“Pharmaceutically acceptable” refers to approved or approvable by a regulatory agency of a federal or a state government or listed in the U.S. Pharmacopoeia or other generally recognized pharmacopoeia for use in animals, and more particularly in humans.

“Pharmaceutically acceptable salt” refers to a salt of a compound, which possesses the desired pharmacological activity of the parent compound. Such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; and (2) salts formed when an acidic proton present in the parent compound is replaced by a metal ion, e.g., an alkali metal ion, an alkaline earth ion, or an aluminum ion; or coordinates with an organic base such as ethanolamine, diethanolamine, triethanolamine, N-methylglucamine, and the like.

“Pharmaceutically acceptable vehicle” refers to a pharmaceutically acceptable diluent, a pharmaceutically acceptable adjuvant, a pharmaceutically acceptable excipient, a pharmaceutically acceptable carrier, or a combination of any of the foregoing with which a compound provided by the present disclosure may be administered to a patient and which does not destroy the pharmacological activity thereof and which is nontoxic when administered in doses sufficient to provide a therapeutically effective amount of the compound.

“Pharmaceutical composition” refers to at least one compound of Formula (I) or Formula (II) and at least one pharmaceutically acceptable vehicle, with which the compound is administered to a patient.

“Preventing” or “prevention” refers to suppressing or reducing the likelihood of acquiring tinnitus (i.e., causing at least one of the clinical symptoms of tinnitus not to develop in a patient that may be exposed to a factor believed to cause tinnitus or predisposed to tinnitus but does not yet experience or display symptoms of tinnitus).

“Prodrug” refers to a derivative of a drug molecule that requires a transformation within the body to release the active drug. Prodrugs are frequently, although not necessarily, pharmacologically inactive until converted to the parent drug. Compounds of Formula (I) and Formula (II) are prodrugs of gabapentin or pregabalin that may be metabolized within a patient's body to form the corresponding GABA analog parent drug.

“Promoiety” refers to a form of protecting group that when used to mask a functional group of a drug molecule converts the drug into a prodrug. For example, the promoiety may be attached to the drug via bond(s) that are cleaved by enzymatic or non-enzymatic means in vivo.

“Protecting group” refers to a grouping of atoms, which when attached to a reactive group in a molecule masks, reduces, or prevents that reactivity. Examples of protecting groups can be found in Green et al., “Protective Groups in Organic Chemistry,” (Wiley, 2^{nd} ed. 1991) and Harrison et al., “Compendium of Synthetic Organic Methods,” Vols. 1-8 (John Wiley and Sons, 1971-1996). Examples of amino protecting groups include, but are not limited to, formyl, acetyl, trifluoroacetyl, benzyl, benzyloxycarbonyl (CBZ), tert-butoxycarbonyl (Boc), trimethylsilyl (TMS), 2-trimethylsilyl-ethanesulfonyl (SES), trityl and substituted trityl groups, allyloxycarbonyl, 9-fluorenylmethyloxycarbonyl (FMOC), nitro-veratryloxycarbonyl (NVOC), and the like. Examples of hydroxy protecting groups include, but are not limited to, those in which the hydroxy group is either acylated or alkylated such as benzyl, and trityl ethers as well as alkyl ethers, tetrahydropyranyl ethers, trialkylsilyl ethers, and allyl ethers.

“Solvate” refers to a molecular complex of a compound with one or more solvent molecules in a stoichiometric or non-stoichiometric amount. Such solvent molecules are those commonly used in the pharmaceutical art, which are known to be innocuous to a patient, e.g., water, ethanol, and the like. A molecular complex of a compound or moiety of a compound and a solvent may be stabilized by non-covalent intra-molecular forces such as, for example, electrostatic forces, van der Waals forces, or hydrogen bonds. The term “hydrate” refers to a solvate in which the one or more solvent molecules are water including monohydrates and hemi-hydrates.

“Substituted” refers to a group in which one or more hydrogen atoms are independently replaced with the same or different substituent(s). Examples of substituents include, but are not limited to, -M, —R⁶⁰, —O⁻, ═O, —OR⁶⁰, —SR⁶⁰, —S⁻, ═S, —NR⁶⁰R⁶¹, ═NR⁶⁰, —CM, —CF₃, —CN, —OCN, —SCN, —NO, —NO₂, ═N₂, —N₃, —S(O)₂O⁻, —S(O)₂OH, —S(O)₂R⁶⁰, —OS(O₂)O⁻, —OS(O)₂R⁶⁰, —P(O)(O⁻)₂, —P(O)(OR⁶⁰)(O⁻), —OP(O)(OR⁶⁰)(OR⁶¹), —C(O)R⁶⁰, —C(S)R⁶⁰, —C(O)OR⁶⁰, —C(O)NR⁶⁰R⁶¹, —C(O)O⁻, —C(S)OR⁶⁰, —NR⁶²C(O)NR⁶⁰R⁶¹, —NR⁶²C(S)NR⁶⁰R⁶¹, —NR⁶²C(NR⁶³)NR⁶⁰R⁶¹, and —C(NR⁶²)NR⁶⁰R⁶¹ where M is independently a halogen; R⁶⁰, R⁶¹, R⁶², and R⁶³ are independently chosen from hydrogen, alkyl, substituted alkyl, alkoxy, substituted alkoxy, cycloalkyl, substituted cycloalkyl, cycloheteroalkyl, substituted cycloheteroalkyl, aryl, substituted aryl, heteroaryl, and substituted heteroaryl, or R⁶⁰ and R⁶¹ together with the nitrogen atom to which they are bonded form a cycloheteroalkyl or substituted cycloheteroalkyl ring.

In certain embodiments, each substituent group is independently chosen from halogen, —NH₂, —OH, —CN, —CF₃, —COOH, —C(O)NH₂, —C(O)OR⁶⁴, and —NR⁶⁴₃⁺, wherein each R⁶⁴ is independently C₁₋₃ alkyl.

“Sustained release” refers to release of a compound from a pharmaceutical composition dosage form at a rate effective to achieve a therapeutic or prophylactic concentration of the compound or active metabolite thereof, in the systemic circulation of a patient over a prolonged period of time relative to that achieved by administration of an immediate release formulation of the same compound by the same route of administration. In some embodiments, release of a compound occurs over a time period of at least about 4 hours, such as at least about 8 hours, at least about 12 hours, at least about 16 hours, at least bout 20 hours, and in some embodiments, at least about 24 hours.

“Treating” or “treatment” of tinnitus refers to arresting or ameliorating tinnitus, or at least one of the clinical symptoms of tinnitus, reducing the risk of acquiring tinnitus, or at least one of the clinical symptoms of tinnitus, reducing the development of tinnitus or at least one of the clinical symptoms of tinnitus, or reducing the risk of developing tinnitus, or at least one of the clinical symptoms of tinnitus. “Treating” or “treatment” also refers to inhibiting tinnitus, either physically, (e.g., suppressing, reducing, or stabilizing a discernible symptom), physiologically, (e.g., suppressing, reducing, or stabilizing a physical parameter), or both, and to inhibiting at least one physical parameter that may or may not be discernible to the patient. In certain embodiments, “treating” or “treatment” refers to delaying the onset of tinnitus or at least one or more symptoms thereof in a patient which may be exposed to a factor believed to cause tinnitus or predisposed to tinnitus even though that patient does not yet experience or display symptoms of tinnitus.

The terms “treating” and “treatment” and “to treat” refer to preventing, reducing, or eliminating tinnitus and/or the accompanying symptoms of tinnitus in a patient. Treatment refers to any indicia of success in prevention, reduction, elimination, or amelioration of tinnitus, including any objective or subjective parameter such as abatement, remission, diminishing of symptoms, prevention, or lessening of tinnitus symptoms or making the condition more tolerable to the patient, making the tinnitus less debilitating, or improving a patient's physical or mental well-being. For example, success of treatment by methods of treating tinnitus provided by the present disclosure may be measured by comparing the frequency and/or severity of tinnitus before treatment with a prodrug of Formula (I) or Formula (II) is initiated, with the frequency and/or severity of tinnitus following the initiation of treatment. The prevention, treatment, or amelioration of tinnitus symptoms may be based on objective or subjective parameters, including the results of a physical examination, or personal interview regarding symptom severity and quality of life, or any other appropriate means known in the art.

“Therapeutically effective amount” refers to the amount of a compound that, when administered to a subject for treating tinnitus, or at least one of the clinical symptoms of tinnitus, is sufficient to affect such treatment of tinnitus or symptom thereof. The “therapeutically effective amount” may vary depending, for example, on the compound, the etiology of the tinnitus, severity of the tinnitus and/or symptoms thereof, the age, weight, and/or health of the patient to be treated, and the judgment of the prescribing physician. An appropriate amount in any given instance may be ascertained by those skilled in the art or capable of determination by routine experimentation.

“Therapeutically effective dose” refers to a dose that provides effective treatment of tinnitus in a patient. A therapeutically effective dose may vary from compound to compound, and from patient to patient, and may depend upon factors such as the condition of the patient and the route of delivery. A therapeutically effective dose may be determined in accordance with routine pharmacological procedures known to those skilled in the art.

Reference is now made in detail to certain embodiments of pharmaceutical compositions and methods provided by the present disclosure. The disclosed embodiments are not intended to be limiting of the claims. To the contrary, the claims are intended to cover all alternatives, modifications, and equivalents.

### Prodrugs of Gabapentin and Pregabalin

In certain embodiments, a prodrug of gabapentin or pregabalin is chosen from compounds of Formula (I) and Formula (II):

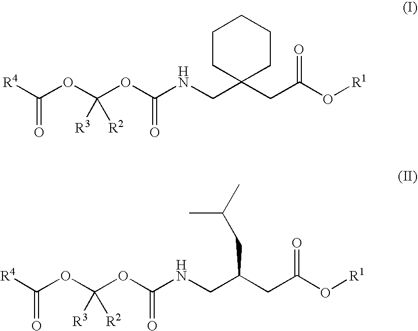

a pharmaceutically acceptable salt thereof, a pharmaceutically acceptable solvate of any of the foregoing, and a pharmaceutically acceptable N-oxide of any of the foregoing, wherein:

R¹ is selected from hydrogen, alkyl, substituted alkyl, aryl, substituted aryl, arylalkyl, substituted arylalkyl, cycloalkyl, substituted cycloalkyl, cycloheteroalkyl, substituted cycloheteroalkyl, heteroalkyl, substituted heteroalkyl, heteroaryl, substituted heteroaryl, heteroarylalkyl, and substituted heteroarylalkyl;

R² and R³ are independently chosen from hydrogen, alkyl, substituted alkyl, alkoxycarbonyl, substituted alkoxycarbonyl, aryl, substituted aryl, arylalkyl, substituted arylalkyl, carbamoyl, substituted carbamoyl, cycloalkyl, substituted cycloalkyl, heteroalkyl, substituted heteroalkyl, cycloheteroalkyl, substituted cycloheteroalkyl, heteroaryl, substituted heteroaryl, heteroarylalkyl, and substituted heteroarylalkyl, or R² and R³ together with the carbon atom to which they are bonded form a cycloalkyl, substituted cycloalkyl, cycloheteroalkyl, or substituted cycloheteroalkyl ring; and

R⁴ is chosen from acyl, substituted acyl, alkyl, substituted alkyl, aryl, substituted aryl, arylalkyl, substituted arylalkyl, cycloalkyl, substituted cycloalkyl, cycloheteroalkyl, substituted cycloheteroalkyl, heteroalkyl, substituted heteroalkyl, heteroaryl, substituted heteroaryl, heteroarylalkyl, and substituted heteroarylalkyl.

In certain embodiments, for example, when R⁴ is substituted alkyl, each substituent group is independently chosen from halogen, —NH₂, —OH, —CN, —CF₃, —COOH, —C(O)NH₂, —C(O)OR⁵, and —NR⁵₃⁺ wherein each R⁵ is independently C₁₋₃ alkyl.

In certain embodiments of compounds of Formulae (I) and (II), R¹ is hydrogen.

In certain embodiments of compounds of Formulae (I) and (II), at least one of R² and R³ is other than hydrogen.

In certain embodiments of compounds of Formulae (I) and (II), R² and R³ are independently chosen from hydrogen and C₁₋₆ alkyl.

In certain embodiments of compounds of Formulae (I) and (II), R³ is chosen from methyl, ethyl, n-propyl, isopropyl, n-butyl, isobutyl, and sec-butyl, and R² is hydrogen.

In certain embodiments of compounds of Formulae (I) and (II), R³ is chosen from methyl, ethyl, n-propyl, and isopropyl, and R² is hydrogen.

In certain embodiments of compounds of Formulae (I) and (II), R⁴ is chosen from C₁₋₆ alkyl and C₁₋₆ substituted alkyl. In certain embodiments of compounds of Formulae (I) and (II) wherein R⁴ is chosen from C₁₋₆ substituted alkyl, the substituent group is chosen from halogen, —NH₂, —OH, —CN, —CF₃, —COOH, —C(O)NH₂, —C(O)OR⁵, and —NR⁵₃⁺ wherein each R⁵ is independently C₁₋₃ alkyl.

In certain embodiments of compounds of Formulae (I) and (II), R⁴ is chosen from methyl, ethyl, n-propyl, isopropyl, n-butyl, isobutyl, sec-butyl, n-pentyl, isopentyl, sec-pentyl, neopentyl, and 1,1-diethoxyethyl.

In certain embodiments of compounds of Formulae (I) and (II), R⁴ is chosen from methyl, ethyl, n-propyl, isopropyl, n-butyl, and isobutyl.

In certain embodiments of compounds of Formulae (I) and (II), R¹ and R² are each hydrogen, R³ is C₁₋₆ alkyl, and R⁴ is chosen from C₁₋₆ alkyl and C₁₋₆ substituted alkyl. In certain embodiments of compounds of Formulae (I) and (II), wherein R¹ and R² are each hydrogen, R³ is C₁₋₆ alkyl, and R⁴ is chosen from C₁₋₆ substituted alkyl, each substituent group is independently chosen from halogen, —NH₂, —OH, —CN, —CF₃, —COOH, —C(O)NH₂, —C(O)OR⁵, and —NR⁵₃⁺ wherein each R⁵ is independently C₁₋₃ alkyl.

In certain embodiments of compounds of Formulae (I) and (II), R¹ and R² are each hydrogen, R³ is chosen from methyl, ethyl, n-propyl, isopropyl, n-butyl, isobutyl, and sec-butyl, and R⁴ is chosen from methyl, ethyl, n-propyl, isopropyl, n-butyl, isobutyl, sec-butyl, n-pentyl, isopentyl, sec-pentyl, neopentyl, and 1,1-diethoxyethyl.

In certain embodiments of compounds of Formulae (I) and (II), R¹ and R² are each hydrogen, R³ is chosen from methyl, ethyl, n-propyl, and isopropyl, and R⁴ is chosen from methyl, ethyl, n-propyl, isopropyl, n-butyl, and isobutyl.

In certain embodiments, the compound of Formula (I) where R⁴ is isopropyl, R² is hydrogen, and R³ is methyl, is 1-{[α-isobutanoyloxyethoxy)carbonyl]aminomethyl}-1-cyclohexane acetic acid or a pharmaceutically acceptable salt thereof, a pharmaceutically acceptable solvate of any of the foregoing, or a pharmaceutically acceptable N-oxide of any of the foregoing.

In certain embodiments, the compound of Formula (I) where R⁴ is isopropyl, R² is hydrogen, and R³ is methyl, is a crystalline form of 1-{[(α-Isobutanoyloxyethoxy)carbonyl]aminomethyl}-1-cyclohexane acetic acid as disclosed in Estrada et al., U.S. Application Publication No. 2005/015405, which is incorporated herein by reference in its entirety. In certain embodiments, crystalline 1-{[(α-isobutanoyloxyethoxy)carbonyl]aminomethyl}-1-cyclohexane acetic acid has characteristic absorption peaks at 7.0°±0.3°, 8.2°±0.3°, 10.5°±0.3°, 12.8°±0.3°, 14.9°±0.30, 16.4°±0.30, 17.9°±0.30, 18.1°±0.3°, 18.9°±0.3°, 20.9°±0.3°, 23.3°±0.3°, 25.3°±0.3°, and 26.6°±0.30 in an X-ray powder diffractogram. In certain embodiments, crystalline 1-{[(α-isobutanoyloxyethoxy)carbonyl]aminomethyl}-1-cyclohexane acetic acid has a melting point range from about 63° C. to about 64° C., in certain embodiments, from about 64° C. to about 66° C., and in certain embodiments, from about 63° C. to about 66° C.

Examples of compounds of Formula (I) include: 1-{[α-acetoxyethoxy)carbonyl]aminomethyl}-1-cyclohexane acetic acid, 1-{[α-propanoyloxyethoxy)carbonyl]aminomethyl}-1-cyclohexane acetic acid, 1-{[α-butanoyloxyethoxy)carbonyl]aminomethyl}-1-cyclohexane acetic acid, 1-{[α-isobutanoyloxyethoxy)carbonyl]aminomethyl}-1-cyclohexane acetic acid, 1-{[(α-pivaloxyethoxy)carbonyl]aminomethyl}-1-cyclohexane acetic acid, 1-{[α-acetoxymethoxy)carbonyl]aminomethyl}-1-cyclohexane acetic acid, 1-{[α-propanoyloxymethoxy)carbonyl]aminomethyl}-1-cyclohexane acetic acid, 1-{[α-butanoyloxymethoxy)carbonyl]aminomethyl}-1-cyclohexane acetic acid, 1-{[α-isobutanoyloxymethoxy)carbonyl]aminomethyl}-1-cyclohexane acetic acid, 1-{[α-pivaloxymethoxy)carbonyl]aminomethyl}-1-cyclohexane acetic acid, 1-{[α-acetoxypropoxy)carbonyl]aminomethyl}-1-cyclohexane acetic acid, 1-{[α-propanoyloxypropoxy)carbonyl]aminomethyl}-1-cyclohexane acetic acid, 1-{[α-butanoyloxypropoxy)carbonyl]aminomethyl}-1-cyclohexane acetic acid, 1-{[α-isobutanoyloxypropoxy)carbonyl]aminomethyl}-1-cyclohexane acetic acid, 1-{[α-pivaloxypropoxy)carbonyl]aminomethyl}-1-cyclohexane acetic acid, 1-{[α-acetoxyisopropoxy)carbonyl]aminomethyl}-1-cyclohexane acetic acid, 1-{[α-propanoyloxyisopropoxy)carbonyl]aminomethyl}-1-cyclohexane acetic acid, 1-{[α-butanoyloxyisopropoxy)carbonyl]aminomethyl}-1-cyclohexane acetic acid, 1-{[α-isobutanoyloxyisopropoxy)carbonyl]aminomethyl}-1-cyclohexane acetic acid, 1-{[α-pivaloxyisopropoxy)carbonyl]aminomethyl}-1-cyclohexane acetic acid, 1-{[α-acetoxybutoxy)carbonyl]aminomethyl}-1-cyclohexane acetic acid, 1-{[α-propanoyloxybutoxy)carbonyl]aminomethyl}-1-cyclohexane acetic acid, 1-{[α-butanoyloxybutoxy)carbonyl]aminomethyl}-1-cyclohexane acetic acid, 1-{[α-isobutanoyloxybutoxy)carbonyl]aminomethyl}-1-cyclohexane acetic acid, and 1-{[α-pivaloxybutoxy)carbonyl]aminomethyl}-1-cyclohexane acetic acid, and pharmaceutically acceptable salts thereof, pharmaceutically acceptable solvates of any of the foregoing, and pharmaceutically acceptable N-oxides of any of the foregoing.

Examples of compounds of Formula (II) include: 3-{[α-acetoxyethoxy)carbonyl]aminomethyl}-5-methyl hexanoic acid, 3-{[α-propanoyloxyethoxy)carbonyl]aminomethyl}-5-methyl hexanoic acid, 3-{[α-butanoyloxyethoxy)carbonyl]aminomethyl}-5-methyl hexanoic acid, 3-{[α-isobutanoyloxyethoxy)carbonyl]aminomethyl}-5-methyl hexanoic acid, 3-{[α-pivaloxyethoxy)carbonyl]aminomethyl}-5-methyl hexanoic acid, 3-{[α-acetoxymethoxy)carbonyl]aminomethyl}-5-methyl hexanoic acid, 3-{[(α-propanoyloxymethoxy)carbonyl]aminomethyl}-5-methyl hexanoic acid, 3-{[α-butanoyloxymethoxy)carbonyl]aminomethyl}-5-methyl hexanoic acid, 3-{[α-isobutanoyloxymethoxy)carbonyl]aminomethyl}-5-methyl hexanoic acid, 3-{[α-pivaloxymethoxy)carbonyl]aminomethyl}-5-methyl hexanoic acid, 3-{[α-acetoxypropoxy)carbonyl]aminomethyl}-5-methyl hexanoic acid, 3-{[α-propanoyloxypropoxy)carbonyl]aminomethyl}-5-methyl hexanoic acid, 3-{[α-butanoyloxypropoxy)carbonyl]aminomethyl}-5-methyl hexanoic acid, 3-{[α-isobutanoyloxypropoxy)carbonyl]aminomethyl}-5-methyl hexanoic acid, 3-{[α-pivaloxypropoxy)carbonyl]aminomethyl}-5-methyl hexanoic acid, 3-{[α-acetoxyisopropoxy)carbonyl]aminomethyl}-5-methyl hexanoic acid, 3-{[α-propanoyloxyisopropoxy)carbonyl]aminomethyl}-5-methyl hexanoic acid, 3-{[α-butanoyloxyisopropoxy)carbonyl]aminomethyl}-5-methyl hexanoic acid, 3-{[α-isobutanoyloxyisopropoxy)carbonyl]aminomethyl}-5-methyl hexanoic acid, 3-{[α-pivaloxyisopropoxy)carbonyl]aminomethyl}-5-methyl hexanoic acid, 3-{[α-acetoxybutoxy)carbonyl]aminomethyl}-5-methyl hexanoic acid, 3-{[α-propanoyloxybutoxy)carbonyl]aminomethyl}-5-methyl hexanoic acid, 3-{[α-butanoyloxybutoxy)carbonyl]aminomethyl}-5-methyl hexanoic acid, 3-{[α-isobutanoyloxybutoxy)carbonyl]aminomethyl}-5-methyl hexanoic acid, and 3-{[α-pivaloxybutoxy)carbonyl]aminomethyl}-5-methyl hexanoic acid, and pharmaceutically acceptable salts thereof, pharmaceutically acceptable solvates of any of the foregoing, and pharmaceutically acceptable N-oxides of any of the foregoing.

In certain embodiments, the compound of Formula (II) is 3-{[α-isobutanoyloxyethoxy)carbonyl]aminomethyl}-5-methyl hexanoic acid, a pharmaceutically acceptable salt thereof, a pharmaceutically acceptable solvate of any of the foregoing, or a pharmaceutically acceptable N-oxide of any of the foregoing.

### Methods of Synthesis of Prodrugs of Gabapentin and Pregabalin

Methods of synthesis of prodrugs of gabapentin and pregabalin, including methods of synthesizing compounds of structural Formula (I) and Formula (II) are disclosed in Gallop et al., PCT International Publication No. WO 02/100347; Gallop et al., U.S. Application Publication No. 2004/0077553; and Bhat et al., U.S. Application Publication No. 2005/0070715, each of which is incorporated by reference herein in its entirety.

### Methods of Use

In certain embodiments, a prodrug of Formula (I) or Formula (II) or pharmaceutical composition thereof may be administered to a patient suffering from tinnitus. In certain embodiments, a prodrug of Formula (I) or Formula (II) or pharmaceutical composition thereof may be administered to a patient as a preventive measure against tinnitus. The suitability of prodrugs of Formula (I) or Formula (II), or pharmaceutical compositions thereof to treat or prevent tinnitus may be determined by methods known to those skilled in the art.

When used in the present methods of treatment, upon releasing a prodrug of Formula (I) or Formula (II) in vivo, a dosage form comprising a prodrug of Formula (I) or Formula (II) or pharmaceutical composition thereof may provide the GABA analog (e.g., gabapentin or pregabalin) in the systemic circulation of a patient. The promoiety or promoieties of the prodrug may be cleaved either chemically and/or enzymatically. One or more enzymes present in the stomach, intestinal lumen, intestinal tissue, blood, liver, brain, or any other suitable tissue of a mammal may cleave the promoiety or promoieties of the prodrug. The mechanism of cleavage is not important to the current methods. In certain embodiments, gabapentin or pregabalin that is formed by cleavage of the promoiety from the corresponding GABA analog prodrug does not contain substantial quantities of lactam contaminant (such as, less than about 0.5% by weight, for example, less than about 0.2% by weight, and in certain embodiments, less than about 0.1% by weight) for the reasons described in Augart et al., U.S. Pat. No. 6,054,482. The extent of release of lactam contaminant from a prodrug of Formula (I) or Formula (II) may be assessed using standard in vitro analytical methods.

Some therapeutically effective GABA analogs, e.g., gabapentin and pregabalin, have poor passive permeability across the gastrointestinal mucosa, possibly because of their zwitterionic character at physiological pH. Gabapentin, pregabalin, and other GABA analogs are actively transported across the gastrointestinal tract by one or more amino acid transporters (e.g., the “large neutral amino acid transporter”). However, the large neutral amino acid transporter is expressed predominantly within cells lining the lumen of a limited region of the small intestine, which provides a limited window for drug absorption and leads to an overall dose-dependent drug bioavailability that decreases with increasing dose.

The compounds disclosed herein, for example the gabapentin prodrug 1-{[(α-isobutanoyloxyethoxy)carbonyl]aminomethyl}-1-cyclohexane acetic acid, may be more efficacious than the parent drug molecule (e.g., gabapentin or other GABA analog) in treating or preventing tinnitus because the disclosed compounds require less time to reach a therapeutic concentration in the systemic circulation, i.e., the compounds disclosed herein have a shorter Tₘₐₓ than their parent drug counterparts when taken orally. It is believed that the compounds disclosed herein, for example, the gabapentin prodrug 1-{[(α-isobutanoyloxyethoxy)carbonyl]aminomethyl}-1-cyclohexane acetic acid, are absorbed from the gastrointestinal lumen into the blood by a different mechanism than that by which gabapentin and other known GABA analogs are absorbed. For example, gabapentin is believed to be actively transported across the gut wall by a carrier transporter localized in the human small intestine. The gabapentin transporter is easily saturated which means that the amount of gabapentin absorbed into the blood may not be proportional to the amount of gabapentin that is administered orally, since once the transporter is saturated, further absorption of gabapentin does not occur to any significant degree. In comparison to gabapentin, the compounds disclosed herein, for example, the gabapentin prodrug 1-{[(α-isobutanoyloxyethoxy)carbonyl]aminomethyl}-1-cyclohexane acetic acid, are believed to be absorbed across the gut wall along a greater portion of the gastrointestinal tract, including the colon.

Because the compounds disclosed herein may be effectively formulated in sustained release formulations, which provide for sustained release of a GABA analog prodrug into the gastrointestinal tract, for example, within the colon, over a period of hours, the compounds, such as the gabapentin prodrug 1-{[(α-isobutanoyloxyethoxy)carbonyl]aminomethyl}-1-cyclohexane acetic acid, may be more efficacious than their respective parent drugs (e.g., gabapentin or other GABA analog) in treating or preventing tinnitus. The ability of the compounds provided by the present disclosure to be used in sustained release oral dosage forms may reduce the dosing frequency necessary for maintenance of a therapeutically effective drug concentration in the systemic circulation.

A dosage form comprising a prodrug of Formula (I) or Formula (II) may be administered or applied singly or in combination with other agents. The dosage forms may also deliver a prodrug of Formula (I) or Formula (II) to a patient in combination with another pharmaceutically active agent including another prodrug of a GABA analog and/or another active agent known or believed to be capable of treating or preventing tinnitus.

In certain embodiments, a prodrug of Formula (I) or Formula (II) may be suitable for oral administration. In certain embodiments, the promoiety or promoieties are cleaved after absorption of the GABA analog prodrug by the gastrointestinal tract (e.g., in intestinal tissue, blood, liver or other suitable tissue of the patient) following oral administration of the GABA analog prodrug. The promoiety or promoieties may make the prodrug a substrate for one or more transporters expressed in the large intestine (i.e., colon), and/or, for GABA analogs that are poorly absorbed across the gastrointestinal mucosa (e.g., gabapentin and pregabalin), may facilitate the ability of the prodrug to be passively absorbed across the gastrointestinal mucosa.

GABA analog prodrugs of Formula (I) or Formula (II) or pharmaceutical composition thereof may be administered to a patient prior to, during or after tinnitus manifests.

The ability of GABA analog prodrugs of Formula (I) or Formula (II) to treat tinnitus in animal models may be evaluated using the method described by Bauer and Brozoski, J Assoc Res. Otolaryngology 2001, 2(1), 54-64, in which tinnitus is induced by acoustic trauma and by the method described in Guitton et al., J Neuroscience 2003, 23(9), 3944-3952 in which tinnitus is induced by salicylate administration (also see U.S. Application Publication No. 2006/0063802) or by any other suitable method known to those skilled in the art. The ability of a compound to treat tinnitus in human patients may be assessed using objective and subjective tests such as those described in Bauer and Brozoski, Laryngoscope 2006, 116(5), 675-681. An example of a test used in a clinical context to assess tinnitus treatment outcomes is the Tinnitus Handicap Inventory (Newman et al., Arch Otolaryngol Head Neck Surg 1996, 122(2), 143-8).

### Pharmaceutical Compositions

Pharmaceutical compositions provided by the present disclosure comprise at least one GABA analog prodrug of Formula (I) and/or Formula (II) and a pharmaceutically acceptable vehicle. A pharmaceutical composition may comprise a therapeutically effective amount of a prodrug of Formula (I) and/or Formula (II) and at least one pharmaceutically acceptable vehicle. In certain embodiments, a pharmaceutical composition may include more than one prodrug of Formula (I) and/or Formula (II). Pharmaceutically acceptable vehicles include diluents, adjuvants, excipients, and carriers.

Pharmaceutical compositions may be produced using standard procedures. Pharmaceutical compositions may be manufactured by means of conventional mixing, dissolving, granulating, dragee-making, levigating, emulsifying, encapsulating, entrapping, or lyophilizing processes. Pharmaceutical compositions may be formulated in a conventional manner using one or more physiologically acceptable carriers, diluents, excipients, or auxiliaries, which facilitate processing of compounds disclosed herein into preparations, which may be used pharmaceutically. Proper formulation may depend, in part, on the route of administration

Pharmaceutical compositions provided by the present disclosure may provide therapeutic or prophylactic levels of gabapentin or pregabalin for treating tinnitus upon administration to a patient. The promoiety of a GABA analog prodrug may be cleaved in vivo either chemically and/or enzymatically to release the corresponding a GABA analog. One or more enzymes present in the stomach, intestinal lumen, intestinal tissue, blood, liver, brain, or any other suitable tissue of a mammal may enzymatically cleave the promoiety of the administered prodrugs. For example, the promoiety may be cleaved prior to absorption by the gastrointestinal tract (e.g., within the stomach or intestinal lumen) and/or after absorption by the gastrointestinal tract (e.g., in intestinal tissue, blood, liver, or other suitable tissue of a mammal). In certain embodiments, a GABA analog remains conjugated to the promoiety during transit across the intestinal mucosal barrier to provide protection from presystemic metabolism. In certain embodiments, a prodrug of Formula (I) or Formula (II) is essentially not metabolized to release the corresponding GABA analog within enterocytes, but is metabolized to the parent drug within the systemic circulation. Cleavage of the promoiety of a GABA analog prodrug after absorption by the gastrointestinal tract may allow the prodrug to be absorbed into the systemic circulation either by active transport, passive diffusion, or by a combination of both active and passive processes.

GABA analog prodrugs of Formula (I) or Formula (II) may remain intact until after passage of the prodrug through a biological barrier, such as the blood-brain-barrier. In certain embodiments, prodrugs provided by the present disclosure may be partially cleaved, e.g., one or more, but not all, of the promoieties may be cleaved before passage through a biological barrier or prior to being taken up by a cell, tissue, or organ.

GABA analog prodrugs of Formula (I) or Formula (II) may remain intact in the systemic circulation and be absorbed by cells of an organ, either passively or by active transport mechanisms. In certain embodiments, a GABA analog prodrug will be lipophilic and may passively translocate through cellular membranes. Following cellular uptake, the prodrug may be cleaved chemically and/or enzymatically to release the corresponding GABA analog into the cellular cytoplasm, resulting in an increase in the intracellular concentration of the GABA analog.

In certain embodiments, a pharmaceutical composition may comprise at least one prodrug of Formula (I) or Formula (II) in an amount effective for the treatment or prevention of tinnitus in a patient.

In certain embodiments, a pharmaceutical composition may include an adjuvant that facilitates absorption of a GABA analog prodrug of Formula (I) or Formula (II) through the gastrointestinal epithelia. Such enhancers can, for example, open the tight-junctions in the gastrointestinal tract or modify the effect of cellular components, such as p-glycoprotein and the like. Suitable enhancers may include alkali metal salts of salicylic acid, such as sodium salicylate, caprylic or capric acid, such as sodium caprylate or sodium caprate, and the like. Enhancers may include, for example, bile salts, such as sodium deoxycholate. Various p-glycoprotein modulators are described in Fukazawa et al., U.S. Pat. No. 5,112,817; and Pfister et al., U.S. Pat. No. 5,643,909. Various absorption enhancing compounds and materials are described in Burnside et al., U.S. Pat. No. 5,824,638; and Meezam et al., U.S. Application Publication No. 2006/0046962. Other adjuvants that enhance permeability of cellular membranes include resorcinol, surfactants, polyethylene glycol, and bile acids.

In certain embodiments, a pharmaceutical composition may include an adjuvant that reduces enzymatic degradation of a prodrug of Formula (I) or Formula (II). Microencapsulation using protenoid microspheres, liposomes, or polysaccharides may also be effective in reducing enzymatic degradation of administered compounds.

A pharmaceutical composition may also include one or more pharmaceutically acceptable vehicles, including excipients, adjuvants, carriers, diluents, binders, lubricants, disintegrants, colorants, stabilizers, surfactants, fillers, buffers, thickeners, emulsifiers, wetting agents, and the like. Vehicles may be selected, for example, to alter the porosity and permeability of a pharmaceutical composition, alter hydration and disintegration properties, control hydration, enhance manufacturability, etc.

In certain embodiments, a pharmaceutical composition may be formulated for oral administration. Pharmaceutical compositions formulated for oral administration may provide for uptake of a prodrug of Formula (I) or Formula (II) throughout the gastrointestinal tract, or in a particular region or regions of the gastrointestinal tract. In certain embodiments, a pharmaceutical composition may be formulated to enhance uptake a prodrug of Formula (I) or Formula (II) from the lower gastrointestinal tract, and in certain embodiments, from the large intestine, including the colon. Such compositions may be prepared in a manner known in the pharmaceutical art and may further comprise, in addition to a prodrug of Formula (I) or Formula (II), one or more pharmaceutically acceptable vehicles, permeability enhancers, and/or a second therapeutic agent.

In certain embodiments, a pharmaceutical composition may further comprise substances to enhance, modulate and/or control release, bioavailability, therapeutic efficacy, therapeutic potency, stability, and the like. For example, to enhance therapeutic efficacy a prodrug of Formula (I) or Formula (II) may be co-administered with one or more active agents to increase the absorption or diffusion of a prodrug of Formula (I) or Formula (II) from the gastrointestinal tract, or to inhibit degradation of the drug in the systemic circulation. In certain embodiments, a prodrug of Formula (I) or Formula (II) may be co-administered with active agents having pharmacological effects that enhance the therapeutic efficacy of a prodrug of Formula (I) or Formula (II).

Pharmaceutical compositions may take the form of solutions, suspensions, emulsions, tablets, pills, pellets, capsules, capsules containing liquids, powders, sustained-release formulations, suppositories, emulsions, aerosols, sprays, suspensions, liquids, gels or any other form suitable for use.

Pharmaceutical compositions comprising a prodrug of Formula (I) or Formula (II) may be formulated for oral administration. Pharmaceutical compositions for oral delivery may be in the form of tablets, lozenges, aqueous or oily suspensions, granules, powders, emulsions, capsules, syrups, or elixirs, for example. Orally administered compositions may contain one or more optional agents, for example, sweetening agents such as fructose, aspartame or saccharin, flavoring agents such as peppermint, oil of wintergreen, or cherry coloring agents and preserving agents, to provide a pharmaceutically palatable preparation. Moreover, when in tablet or pill form, the compositions may be coated to delay disintegration and absorption in the gastrointestinal tract, thereby providing a sustained action over an extended period of time. Oral compositions may include standard vehicles such as mannitol, lactose, starch, magnesium stearate, sodium saccharine, cellulose, magnesium carbonate, etc. Such vehicles may be of pharmaceutical grade.

For oral liquid preparations such as, for example, suspensions, elixirs, and solutions, suitable carriers, excipients or diluents include water, saline, alkyleneglycols (e.g., propylene glycol), polyalkylene glycols (e.g., polyethylene glycol) oils, alcohols, slightly acidic buffers between pH 4 and pH 6 (e.g., acetate, citrate, ascorbate at between about 5 mM to about 50 mM), etc. Additionally, flavoring agents, preservatives, coloring agents, bile salts, acylcarnitines, and the like may be added.

When a prodrug of Formula (I) or Formula (II) is acidic, it may be included in any of the formulations provided by the present disclosure as the free acid, a pharmaceutically acceptable salt, a solvate, or a hydrate. Pharmaceutically acceptable salts substantially retain the activity of the free acid, may be prepared by reaction with bases, and tend to be more soluble in aqueous and other protic solvents than the corresponding free acid form. In some embodiments, sodium salts of a prodrug of Formula (I) or Formula (II) may be used in a formulation.

Pharmaceutical compositions provided by the present disclosure may be formulated for parenteral administration including administration by injection, for example, into a vein (intravenously), an artery (intraarterially), a muscle (intramuscularly), under the skin (subcutaneously or in a depot formulation), to the pericardium, to the coronary arteries, or used as a solution for delivery to a tissue or organ, for example, use in a cardiopulmonary bypass machine or to bathe transplant tissues or organs. Injectable compositions may be pharmaceutical compositions for any route of injectable administration, including, but not limited to, intravenous, intrarterial, intracoronary, pericardial, perivascular, intramuscular, subcutaneous, intradermal, intraperitoneal, and intraarticular. In certain embodiments, an injectable pharmaceutical composition may be a pharmaceutically appropriate composition for administration directly into the heart, pericardium, or coronary arteries.

Pharmaceutical compositions provided by the present disclosure suitable for parenteral administration may comprise one or more prodrugs of Formula (I) or Formula (II) in combination with one or more pharmaceutically acceptable sterile isotonic aqueous, water-miscible, or non-aqueous vehicles. Pharmaceutical compositions for parenteral use may include substances that increase and maintain drug solubility such as complexing agents and surface acting agents, compounds that make the solution isotonic or near physiological pH such as sodium chloride, dextrose, and glycerin, substances that enhance the chemical stability of a solution such as antioxidants, inert gases, chelating agents, and buffers, substances that enhance the chemical and physical stability, substances that minimize self aggregation or interfacial induced aggregation, substances that minimize protein interaction with interfaces, preservatives including antimicrobial agents, suspending agents, emulsifying agents, and combinations of any of the foregoing. Pharmaceutical compositions for parenteral administration may be formulated as solutions, suspensions, emulsions, liposomes, microspheres, nanosystems, and powder to be reconstituted as solutions.

For prolonged delivery, a pharmaceutical composition may be provided as a depot preparation, for administration by implantation, e.g., subcutaneous, intradermal, or intramuscular injection. Thus, in certain embodiments, a pharmaceutical composition may be formulated with suitable polymeric or hydrophobic materials, e.g., as an emulsion in a pharmaceutically acceptable oil, ion exchange resins, or as a sparingly soluble derivative, e.g., as a sparingly soluble salt form of a prodrug of Formula (I) or Formula (II).

Pharmaceutical compositions comprising a GABA analog prodrug of Formula (I) or Formula (II) apriority for topical administration may be in the form of creams, gels, ointments, patches, pasts, sprays, or viscous lotions. Such formulations may comprise one or more GABA analog prodrugs of Formula (I) or Formula (II), generally in purified form, together with a suitable amount of a pharmaceutically acceptable topical vehicle including, but not limited to, gels, lotions, creams, ointments, and liquids. Topical delivery systems also include transdermal patches containing at least one GABA analog prodrugs of Formula (I) or Formula (II) to be administered. Delivery through the skin may be achieved by diffusion or by more active energy sources such as iontophoresis or electrotransport.

Formulations of a GABA analog prodrug of Formula (I) or Formula (II), for topical use, such as in creams, ointments, and gels, may include an oleaginous or water-soluble ointment base. For example, topical compositions may include vegetable oils, animal fats, and in certain embodiments, semisolid hydrocarbons obtained from petroleum. Topical compositions may further include white ointment, yellow ointment, cetyl esters wax, oleic acid, olive oil, paraffin, petrolatum, white petrolatum, spermaceti, starch glycerite, white wax, yellow wax, lanolin, anhydrous lanolin, and glyceryl monostearate. Various water-soluble ointment bases may also be used, including glycol ethers and derivatives, polyethylene glycols, polyoxyl 40 stearate, and polysorbates.

Pharmaceutical compositions comprising a GABA analog prodrug of Formula (I) or Formula (II) may also be formulated for otic administration. Otic pharmaceutical compositions include solutions, suspensions, and ointments that may be applied within the ear canal and/or to the outer skin of the ear. Solutions or suspensions may be formulated as ear drops comprising at least one compound provided by the present disclosure together with at least one vehicle such as, but not limited to, propylene glycol, anhydrous glycerin, polyethylene glycol, mineral oil, or a combination of any of the foregoing. Otic pharmaceutical compositions for topical application to the external ear may be formulated as aqueous solutions or suspensions, oil-in water emulsions such as described in U.S. Pat. No. 5,753,269, combined with polymeric materials such as U.S. Pat. No. 5,747,061, gel forming compositions such as U.S. Pat. Nos. 6,316,011 and 6,346,272, or combined with low molecular weight polymers such as described in U.S. Application Publication No. 2004/0014819. Topical otic compositions may contain one or more pharmaceutically acceptable components such as surfactants, adjuvants, additional medicaments, buffers, antioxidants, tonicity adjusters, preservatives, thickeners or viscosity modifiers, and the like.

Examples of vehicles for otic pharmaceutical compositions include saline, alcohols, vegetable oils, benzyl alcohols, alkylene glycols, polyethylene glycols, glycerol triacetate, gelatin, carbohydrates such as lactose or starch, magnesium, stearate, talc, and petrolatum. The pharmaceutical compositions may be sterilized and/or may contain additional vehicles such as lubricants, preservatives, stabilizers and/or wetting agents, emulsifiers, agents for controlling osmotic pressure, buffers, colorants, and/or aromatizing substances.

In certain embodiments an otic topical excipient is selected that does not enhance delivery of an active agent to the systemic circulation or to the central nervous system when administered to the ear. For example, an otic topical excipient can have substantial occlusive properties, which enhance percutaneous transmission through the mucosa into the systemic circulation. Examples of occlusive vehicles include hydrocarbon bases, anhydrous absorption bases such as hydrophilic petrolatum and anhydrous lanolin, and water-in-oil emulsion bases such as lanolin and cold cream. In certain embodiments, an otic topical vehicle may be substantially non-occlusive such as for example, water-soluble oil-in-water emulsion bases and water-soluble bases such as polyethylene glycol-based vehicles and aqueous solutions gelled with various agents such as methylcellulose, hydroxyethyl cellulose and hydroxypropylmethylcellulose.

Topical otic compositions may be viscous gels that remain in the ear and release the drug over a period from about 2 to about 12 hours. Examples of gels suitable for otic pharmaceutical compositions include, but are not limited to, poloxamers, hyaluronates, xyloglucans, chitosans, polyesters, poly(lactides), poly(glycolides) or their copolymers PLGA, sucrose acetate isobutyrate, and glycerol monooleate.

Topical otic compositions may be an aqueous polymeric suspension. Examples of suspending agents include dextrans, polyethylene glycols, polyvinyl pyrolidone, polysaccharide gels, cellulosic polymers such as hydroxypropyl methylcellulose, and carboxy-containing polymers such as polymers or copolymer soft acrylic acid.

Fluid topical otic pharmaceutical compositions provided by the present disclosure, including both ointments and suspensions, may have a viscosity that is suitable for the selected route of administration. For example, an eardrop may have a viscosity from about 1,000 centipoise to about 30,000 centipoise. A viscous solution or ribbon form for otic administration may have a viscosity from about 30,000 centipoise to about 100,000 centipoise.

In certain embodiments a pharmaceutical composition comprising a GABA analog prodrug of Formula (I) or Formula (II) may be provided in the form of a depot in contact with the surfaces of the ear. A depot refers to a source of a comprising a GABA analog prodrug of Formula (I) or Formula (II) that is not rapidly removed by the ear clearance mechanisms and thereby allows for continued, sustained high concentrations of a comprising a GABA analog prodrug of Formula (I) or Formula (II) to be present in the fluid on the surfaces of the ear by a single application. In general, it is believed that absorption of a drug is dependent on both the dissolved drug concentration and the contact duration of the external tissue of the drug-containing fluid. As the drug is removed by clearance of the fluid and/or absorption into the ear tissue, more drug is provided, e.g., dissolved, into the replenished fluid from the depot. Depending on the depot, one or two applications may provide a complete dosing regimen. In certain embodiments, depot administration may provide a 6 to 14 day treatment concentration within the otic tissue. A depot may take a variety of forms so long as the GABA analog prodrug of Formula (I) or Formula (II) may be provided in sufficient concentration levels to be therapeutically effective, is releasable, and is not readily removed from the ear. Examples of otic depot forms include aqueous polymeric suspensions, ointments, and solid inserts.

A topical otic dosage form may also be in the form of an insert. An insert comprises a matrix containing a GABA analog prodrug of Formula (I) or Formula (II). For example the matrix may be a polymer and the active agent may be dispersed within the polymer matrix and/or bonded to the polymer matrix.

Examples of otic compositions and dosage forms are disclosed in Bowman et al., U.S. Application Publication No. 2006/0046970; and Ansel's Pharmaceutical Dosage Forms and Drug Delivery Systems, 8^{th} ed., Allen et al., eds, Lippincott Williams & Wilkins, 2005, pp. 563-566.

In certain embodiments, pharmaceutical compositions comprising a GABA analog prodrug of Formula (I) or Formula (II) contain no or only low levels of lactam side products formed by intramolecular cyclization of the GABA analog and/or GABA analog prodrug. In certain embodiments, the compositions are stable to extended storage (for example, greater than one year) without substantial lactam formation (for example, less than about 0.5% lactam by weight, such as, less than about 0.2% lactam by weight, and in certain embodiments, less than about 0.1% lactam by weight).

Pharmaceutical compositions provided by the present disclosure may be formulated so as to provide immediate, sustained, or delayed release of a GABA analog prodrug of Formula (I) or Formula (II) after administration to the patient by employing procedures known in the art. In certain embodiments, a pharmaceutical composition comprising a GABA analog prodrug of Formula (I) or Formula (II) may be formulated for sustained release formulation.

### Dosage Forms

Pharmaceutical compositions provided by the present disclosure may be formulated in a unit dosage form. A unit dosage form refers to a physically discrete unit suitable as a unitary dose for patients undergoing treatment, with each unit containing a predetermined quantity of a GABA analog prodrug of Formula (I) or Formula (II) calculated to produce the intended therapeutic effect. A unit dosage form may be for a single daily dose, 1 to 2 times per day, or one of multiple daily doses, e.g., 2 to 4 times per day. When multiple daily doses are used, the unit dosage may be the same or different for each dose. One or more dosage forms may comprise a dose, which may be administered to a patient at a single point in time or during a time interval.

Pharmaceutical compositions provided by the present disclosure may be used in dosage forms that provide immediate release and/or controlled release of a GABA analog prodrug of Formula (I) or Formula (II). The appropriate type of dosage form may depend on the type or severity of tinnitus being treated or prevented, and on the method of administration. In certain embodiments, a dosage form may be adapted to be administered to a patient no more than twice per day, and in certain embodiments, only once per day. Dosing may be provided alone or in combination with other drugs and may continue as long as required for effective treatment of the disease, disorder, or condition.

Pharmaceutical compositions comprising a GABA analog prodrug of Formula (I) or Formula (II) may be formulated for immediate release for parenteral administration, oral administration, or by any other appropriate route of administration.

Controlled drug delivery systems may be designed to deliver a drug in such a way that the drug level is maintained within a therapeutically effective window and effective and safe blood levels are maintained for a period as long as the system continues to deliver the drug at a particular rate. Controlled drug delivery may produce substantially constant blood levels of a drug as compared to fluctuations observed with immediate release dosage forms administered by the same route of administration. For some drugs, maintaining a constant blood and tissue concentration throughout the course of therapy is the most desirable mode of treatment. Immediate release of these drugs may cause blood levels to peak above the level required to elicit the desired response, which wastes the drug and may cause or exacerbate toxic side effects. Controlled drug delivery may result in optimum therapy, and not only may reduce the frequency of dosing, but may also reduce the severity of side effects. Examples of controlled release dosage forms include dissolution controlled systems, diffusion controlled systems, ion exchange resins, osmotically controlled systems, erodable matrix systems, pH independent formulations, gastric retention systems, and the like.

In certain embodiments, an oral dosage form provided by the present disclosure may be a controlled release dosage form. Controlled delivery technologies may improve the absorption of a drug in a particular region or regions of the gastrointestinal tract. The appropriate oral dosage form for a particular pharmaceutical composition comprising a GABA analog prodrug of Formula (I) or Formula (II) may depend, at least in part, on the gastrointestinal absorption properties of the prodrug of Formula (I) or Formula (II), the stability of the compound of Formula (I) or Formula (II) in the gastrointestinal tract, the pharmacokinetics of the prodrug of Formula (I) or Formula (II), and the intended therapeutic profile. An appropriate controlled release oral dosage form may be selected for a particular the prodrug of Formula (I) or Formula (II). For example, gastric retention oral dosage forms may be appropriate for compounds absorbed primarily from the upper gastrointestinal tract, and sustained release oral dosage forms may be appropriate for compounds absorbed primarily form the lower gastrointestinal tract.

In certain embodiments, pharmaceutical compositions comprising a GABA analog prodrug of Formula (I) or Formula (II) may be practiced with a number of different dosage forms, which may be adapted to provide sustained release of the prodrug of Formula (I) or Formula (II) upon oral administration. Sustained release oral dosage forms may be used to release drugs over a prolonged time period and are useful when it is desired that a drug or drug form be delivered to the lower gastrointestinal tract. Sustained release oral dosage forms include diffusion-controlled systems such as reservoir devices and matrix devices, dissolution-controlled systems, osmotic systems, and erosion-controlled systems. Sustained release oral dosage forms and methods of preparing the same are well known in the art (see, for example, “The Science and Practice of Pharmacy,” Lippincott, Williams & Wilkins, 21st edition, 2005, Chapters 46 and 47; Langer, Science 1990, 249, 1527-1533; and Rosoff, “Controlled Release of Drugs,” 1989, Chapter 2).

Sustained release oral dosage forms include any oral dosage form that maintains therapeutic concentrations of a drug in a biological fluid such as the plasma, blood, cerebrospinal fluid, or in a tissue or organ for a prolonged time period. Sustained release oral dosage forms include diffusion-controlled systems such as reservoir devices and matrix devices, dissolution-controlled systems, osmotic systems, and erosion-controlled systems. Sustained release oral dosage forms and methods of preparing the same are well known in the art.

In diffusion-controlled systems, a water-insoluble polymer controls the flow of fluid and the subsequent egress of dissolved drug from the dosage form. Both diffusional and dissolution processes are involved in release of drug from the dosage form. In reservoir devices, a core comprising a drug is coated with the polymer, and in matrix systems, the drug is dispersed throughout the matrix. Cellulose polymers such as ethylcellulose or cellulose acetate may be used in reservoir devices. Examples of materials useful in matrix systems include methacrylates, acrylates, polyethylene, acrylic acid copolymers, polyvinylchloride, high molecular weight polyvinylalcohols, cellulose derivates, and fatty compounds such as fatty acids, glycerides, and carnauba wax.

In dissolution-controlled systems, the rate of dissolution of the drug is controlled by slowly soluble polymers or by microencapsulation. Once the coating is dissolved, the drug becomes available for dissolution. By varying the thickness and/or the composition of the coating or coatings, the rate of drug release may be controlled. In some dissolution-controlled systems, a fraction of the total dose may comprise an immediate-release component. Dissolution-controlled systems include encapsulated/reservoir dissolution systems and matrix dissolution systems. Encapsulated dissolution systems may be prepared by coating particles or granules of drug with slowly soluble polymers of different thickness or by microencapsulation. Examples of coating materials useful in dissolution-controlled systems include gelatin, carnauba wax, shellac, cellulose acetate phthalate, and cellulose acetate butyrate. Matrix dissolution devices may be prepared, for example, by compressing a drug with a slowly soluble polymer carrier into a tablet form.

The rate of release of drug from osmotic pump systems is determined by the inflow of fluid across a semipermeable membrane into a reservoir, which contains an osmotic agent. The drug is either mixed with the agent or is located in a reservoir. The dosage form contains one or more small orifices from which dissolved drug is pumped at a rate determined by the rate of entrance of water due to osmotic pressure. As osmotic pressure within the dosage form increases, the drug is released through the orifice(s). The rate of release is constant and may be controlled within tight limits yielding relatively constant plasma and/or blood concentrations of the drug. Osmotic pump systems may provide a constant release of drug independent of the environment of the gastrointestinal tract. The rate of drug release may be modified by altering the osmotic agent and/or the size of the one or more orifices.

The release of drug from erosion-controlled systems is determined by the erosion rate of a carrier matrix. Drug is dispersed throughout the polymer and the rate of drug release depends on the erosion rate of the polymer. The drug-containing polymer may degrade from the bulk and/or from the surface of the dosage form.

Sustained release oral dosage forms may be in any appropriate form for oral administration, such as, for example, in the form of tablets, pills, or granules. Granules may be filled into capsules, compressed into tablets, or included in a liquid suspension. Sustained release oral dosage forms may additionally include an exterior coating to provide, for example, acid protection, ease of swallowing, flavor, identification, and the like.

In certain embodiments, sustained release oral dosage forms may comprise a therapeutically effective amount of a GABA analog prodrug of Formula (I) or Formula (II) and a pharmaceutically acceptable vehicle. In certain embodiments, a sustained release oral dosage form may comprise less than a therapeutically effective amount of a GABA analog prodrug of Formula (I) or Formula (II) and a pharmaceutically effective vehicle. Multiple sustained release oral dosage forms, each dosage form comprising less than a therapeutically effective amount of a prodrug of Formula (I) or Formula (II), may be administered at a single time or over a period of time to provide a therapeutically effective dose or regimen for treating or preventing tinnitus.

Sustained release oral dosage forms provided by the present disclosure may release a GABA analog prodrug of Formula (I) or Formula (II) from the dosage form to facilitate the ability of the prodrug of Formula (I) or Formula (II) to be absorbed from an appropriate region of the gastrointestinal tract, for example, in the colon. In certain embodiments, a sustained release oral dosage from may release a prodrug of Formula (I) or Formula (II) from the dosage form over a period of at least about 4 hours, at least about 8 hours, at least about 12 hours, at least about 16 hours, at least about 20 hours, and in certain embodiments, at least about 24 hours. In certain embodiments, a sustained release oral dosage form may release a GABA analog prodrug of Formula (I) or Formula (II) from the dosage form in a delivery pattern of from about 0 wt % to about 20 wt % in about 0 to about 4 hours, about 20 wt % to about 50 wt % in about 0 to about 8 hours, about 55 wt % to about 85 wt % in about 0 to about 14 hours, and about 80 wt % to about 100 wt % in about 0 to about 24 hours. In certain embodiments, a sustained release oral dosage form may release a prodrug of Formula (I) or Formula (II) from the dosage form in a delivery pattern of from about 0 wt % to about 20 wt % in about 0 to about 4 hours, about 20 wt % to about 50 wt % in about 0 to about 8 hours, about 55 wt % to about 85 wt % in about 0 to about 14 hours, and about 80 wt % to about 100 wt % in about 0 to about 20 hours. In certain embodiments, a sustained release oral dosage form may release a prodrug of Formula (I) or Formula (II) from the dosage form in a delivery pattern of from about 0 wt % to about 20 wt % in about 0 to about 2 hours, about 20 wt % to about 50 wt % in about 0 to about 4 hours, about 55 wt % to about 85 wt % in about 0 to about 7 hours, and about 80 wt % to about 100 wt % in about 0 to about 8 hours.

Sustained release oral dosage forms comprising a prodrug of Formula (I) or Formula (II) may provide a concentration of the corresponding GABA analog in the plasma, blood, or tissue of a patient over time, following oral administration to the patient. The concentration profile of gabapentin or pregabalin may exhibit an AUC that is proportional to the dose of the corresponding compound of Formula (I) or Formula (II).

Regardless of the specific form of controlled release oral dosage form used, a GABA analog prodrug of Formula (I) or Formula (II) may be released from an orally administered dosage form over a sufficient period of time to provide prolonged therapeutic concentrations of the prodrug of Formula (I) or Formula (II) in the plasma and/or blood of a patient that is effective for treating or preventing tinnitus. Following oral administration, an oral dosage form comprising a prodrug of Formula (I) or Formula (II) may provide a therapeutically effective concentration of the corresponding GABA analog in the plasma and/or blood of a patient for a continuous time period of at least about 4 hours, of at least about 8 hours, for at least about 12 hours, for at least about 16 hours, and in certain embodiments, for at least about 20 hours following oral administration of the dosage form to the patient. The continuous time periods during which a therapeutically effective concentration of gabapentin or pregabalin is maintained may be the same or different. The continuous period of time during which a therapeutically effective plasma concentration of gabapentin or pregabalin is maintained may begin shortly after oral administration or after a time interval.

In certain embodiments, the dosage form may release from about 0 to about 30% of the prodrug in about 0 to about 2 hours, from about 20 to about 50% of the prodrug in about 2 to about 12 hours, from about 50 to about 85% of the prodrug in about 3 to about 20 hours and greater than about 75% of the prodrug in about 5 to about 18 hours. In certain embodiments, a sustained release oral dosage form may provide a concentration profile of gabapentin or pregabalin in the blood and/or plasma of a patient over time, which has an area under the curve (AUC) that is proportional to the dose of the corresponding GABA analog prodrug of Formula (I) or Formula (II) administered, and a maximum concentration Cₘₐₓ. In certain embodiments, the Cₘₐₓ may be less than about 75%, and in certain embodiments, may be less than about 60%, of the Cₘₐₓ obtained from administering an equivalent dose of the compound from an immediate release oral dosage form and the AUC is substantially the same as the AUC obtained from administering an equivalent dose of the prodrug from an immediate release oral dosage form.

In certain embodiments, a dosage form provided by the present disclosure may be administered twice per day, and in certain embodiments, once per day, to provide a therapeutically effective concentration of a GABA analog, e.g., gabapentin or pregabalin, in the systemic circulation of a patient.

Examples of sustained release oral dosage forms of GABA analogs are disclosed in Cundy et al., U.S. Pat. No. 6,833,140, U.S. Application Publication Nos. 2004/0198820 and 2006/0141034, each of which is incorporated by reference herein in its entirety.

### Methods of Administration and Doses

Methods for the treatment or prevention of tinnitus comprise administering a GABA analog prodrug of Formula (I) or Formula (II), or a pharmaceutical composition thereof, to a patient in need of such treatment or prevention.

A GABA analog prodrug of Formula (I) or Formula (II), or a pharmaceutically acceptable salt thereof, or a pharmaceutically acceptable solvate of any of the foregoing, or a pharmaceutical composition thereof may be administered by any appropriate route. Examples of suitable routes of administration include, but are not limited to, intradermal, intramuscular, intraperitoneal, intravenous, subcutaneous, intranasal, epidural, oral, sublingual, intranasal, intracerebral, intravaginal, transdermal, rectally, inhalation, optically, or topically. Administration may be systemic or local. Administration may be bolus injection, continuous infusion, or by absorption through epithelial or mucocutaneous linings, e.g., oral mucosa, rectal, and intestinal mucosa, etc. Administration may be systemic or local. In certain embodiments, a GABA analog prodrug of Formula (I) or Formula (II), or pharmaceutical composition thereof may be administered orally.

In certain embodiments, a GABA analog prodrug of Formula (I) or Formula (II), or pharmaceutical composition thereof may be administered otically. Pharmaceutical compositions comprising a prodrug of Formula (I) and/or Formula (II) formulated for otic administration may be applied as a liquid drop, ointment, a viscous solution or gel, a ribbon, or as a solid. Administration of a compound to the inner ear may be accomplished by various delivery techniques including using devices or drug carriers to transport and/or deliver a GABA analog prodrug of Formula (I) or Formula (II) in a targeted fashion to the membranes of the round or oval window, where it diffuses into the inner ear or is actively infused such as otowicks (see e.g., U.S. Pat. No. 6,120,484), round window catheters (see e.g., U.S. Pat. Nos. 6,045,528 and 6,377,849), or various types of gels, foams, fibrins or other drug carriers, which are placed in the round window niche or the oval widow and loaded with a GABA analog prodrug of Formula (I) or Formula (II) for sustained release, devices which are inserted into the cochlear duct or any other part of the cochlea (see e.g., U.S. Pat. No. 6,309,410), transtympanic injection, in which the middle ear or part of it is filled by a solution or other carriers of the compound (see e.g., Hoffer et al., Otolarynogologic Clin of N.A. 2003, 36(2), 353-58).

In certain embodiments, a GABA analog prodrug of Formula (I) or Formula (II), or a pharmaceutical composition thereof may be delivered to a patient via sustained release dosage forms, for example, via oral sustained release dosage forms. When used to treat or prevent tinnitus a therapeutically effective amount of one or more GABA analog prodrugs of Formula (I) and/or Formula (II) may be administered or applied singly or in combination with other agents. A therapeutically effective amount of one or more GABA analog prodrugs of Formula (I) or Formula (II) may also deliver a GABA analog prodrug provided by the present disclosure in combination with another pharmaceutically active agent, including another compound provided by the present disclosure. For example, in the treatment of a patient suffering from tinnitus, a dosage form comprising a GABA analog prodrug of Formulae (I) and/or (II) may be administered in conjunction with a therapeutic agent known or believed to be capable of treating or preventing tinnitus, at least one symptom of tinnitus, or at least one condition associated with tinnitus.

The amount of GABA analog prodrug of Formula (I) or Formula (II) that will be effective in the treatment or prevention of tinnitus in a patient will depend, in part, on the nature of the condition and may be determined by standard clinical techniques known in the art. In addition, in vitro or in vivo assays may be employed to help identify optimal dosage ranges. A therapeutically effective amount of prodrug of Formula (I) or Formula (II) to be administered may also depend on, among other factors, the subject being treated, the weight of the subject, the severity of the tinnitus, the manner of administration and the judgment of the prescribing physician.

For systemic administration, a therapeutically effective dose may be estimated initially from in vitro assays. For example, a dose may be formulated in animal models to achieve a beneficial circulating composition concentration range. Initial doses may also be estimated from in vivo data, e.g., animal models, using techniques that are known in the art. Such information may be used to more accurately determine useful doses in humans. One having ordinary skill in the art may optimize administration to humans based on animal data.

In some embodiments, an oral sustained release dosage form is adapted to be administered to a patient from one to three times per day. In some embodiments, an oral sustained release dosage forms are adapted to be administered to a patient from one to two times per day. Dosing may be provided alone or in combination with other drugs and may continue as long as required for effective treatment or prevention of tinnitus.

Suitable dosage ranges for oral administration may depend on the potency of gabapentin or pregabalin (once cleaved from the promoiety) and may be from about 0.1 mg to about 300 mg of drug per kilogram body weight per day, for example, from about 1 to about 100 mg/kg-body weight per day. In certain embodiments, a GABA analog prodrug of Formula (I) may be administered to a patient in an amount from about 10 mg-equivalents to about 3,600 mg-equivalents of gabapentin per day, in certain embodiments, from about 200 mg-equivalents to about 2,400 mg-equivalents of gabapentin per day, and in certain embodiments, from about 400 mg-equivalents to about 1,600 mg-equivalents of gabapentin per day, to treat or prevent tinnitus. Certain GABA analogs may be more potent than gabapentin and lower doses may be appropriate for both the cleaved drug and any prodrug (measured on an equivalent molar basis). In certain embodiments, a GABA analog prodrug of Formula (II) may be administered to a patient in an amount from about 10 mg-equivalents to about 1,200 mg-equivalents of pregabalin per day, in certain embodiments, from about 50 mg-equivalents to about 800 mg-equivalents of pregabalin per day, and in certain embodiments, from about 100 mg-equivalents to about 600 mg-equivalents of pregabalin per day to treat or prevent tinnitus. Dosage ranges may be determined by methods known to those skilled in the art.

A dose may be administered in a single dosage form or in multiple dosage forms. When multiple dosage forms are used the amount of compound contained within each dosage form may be the same or different. The amount of a GABA analog prodrug of Formula (I) or Formula (II) contained in a dose may depend on the route of administration and whether the tinnitus in a patient is effectively treated or prevented by acute, chronic, or a combination of acute and chronic administration.

In certain embodiments an administered dose is less than a toxic dose. Toxicity of the compositions described herein may be determined by standard pharmaceutical procedures in cell cultures or experimental animals, e.g., by determining the LD₅₀ (the dose lethal to 50% of the population) or the LD₁₀₀ (the dose lethal to 100% of the population). The dose ratio between toxic and therapeutic effect is the therapeutic index. In certain embodiments, a pharmaceutical composition may exhibit a high therapeutic index. The data obtained from these cell culture assays and animal studies may be used in formulating a dosage range that is not toxic for use in humans. A dose of a pharmaceutical composition comprising a GABA analog prodrug of Formula (I) or Formula (II) may be within a range of circulating concentrations in for example the blood, plasma, or central nervous system, that include the effective dose and that exhibits little or no toxicity. A dose may vary within this range depending upon the dosage form employed and the route of administration utilized. In certain embodiments, an escalating dose may be administered.

The efficacy of administering a GABA analog prodrug of Formula (I) or Formula (II) for treating or preventing tinnitus may be assessed using animal and human models of tinnitus and on clinical results. Methods of evaluating tinnitus in animals and humans are known (see, e.g., Bauer and Brozoski, J Assoc Res Otolaryngology 2001, 2(1), 54-64; Guitton et al., J Neuroscience 2003, 23(9), 3944-3952; Guitton et al., U.S. Application Publication No. 2006/0063802; Bauer and Brozoski, Laryngoscope 2006, 116(5), 675-681; Soderman et al., Otol Neurotol 2001, 22(4), 526-33; Henry et al., Am J Audiol 2005, 14(1), 21-48; and Folmer, BMC Ear, Nose, and Throat Disorders 2002, 2(3), 1-9).

In certain embodiments, oral administration of an oral sustained release dosage form comprising a GABA analog prodrug of Formula (I) or Formula (II) may provide a therapeutically effective concentration of gabapentin or pregabalin, in the blood plasma of a patient for a time period of at least about 4 hours after administration of the dosage form, in certain embodiments, for a time period of at least about 8 hours, and in certain embodiments, for a time period of at least about 12 hours.

GABA analog prodrugs of Formula (I) or Formula (II) or pharmaceutical composition thereof may be administered to a patient in need of tinnitus treatment in a therapeutically effective amount. A therapeutically effective amount refers to a total amount of GABA analog prodrug that results in a detectable change in the severity of the patient's tinnitus symptoms. A therapeutically effective amount may provide a concentration of the GABA analog prodrug that is pharmacologically active and therapeutically effective.

### Combination Therapy

In certain embodiments, GABA analog prodrugs of Formula (I) or Formula (II), or pharmaceutical compositions thereof may be used in combination therapy with at least one other therapeutic agent including a different GABA analog prodrug of Formula (I) or Formula (II). The GABA analog prodrug of Formula (I) or Formula (II), or pharmaceutical composition thereof and the additional therapeutic agent may act additively or, in certain embodiments, synergistically, such that the combination of the therapeutic agents together are, for example, more effective, safer, and/or produce fewer or less severe side effects. In certain embodiments, a GABA analog prodrug of Formula (I), or Formula (II) or a pharmaceutical composition thereof may be administered concurrently with the administration of another therapeutic agent. In certain embodiments, a GABA analog prodrug of Formula (I) or Formula (II) and/or pharmaceutical composition thereof may be administered prior or subsequent to administration of another therapeutic agent and thus may be used in regimens with overlapping schedules. The additional therapeutic agent may be effective for treating tinnitus, may be effective in treating at least one symptom of tinnitus, may be effective in treating a side effect of administering the GABA analog prodrug of Formula (I) or Formula (II) for treating tinnitus, or may be effective for treating a disease, disorder, or condition other than tinnitus. In certain embodiments in which a prodrug of Formula (I) or Formula (II) is administered together with an additional therapeutic agent for treating for preventing tinnitus each of the active agents may be used at lower doses than when used singly.

Methods provided by the present disclosure include administration of one or more GABA analog prodrugs of Formula (I) or Formula (II) or pharmaceutical compositions comprising a GABA analog prodrug of Formula (I) or Formula (II) and one or more other therapeutic agents provided that the combined administration does not inhibit the therapeutic efficacy of the one or more compounds provided by the present disclosure and/or does not produce adverse combination effects.

In certain embodiments, compositions provided by the present disclosure may be administered concurrently with the administration of another therapeutic agent, which may be part of the same pharmaceutical composition or dosage form as, or in a different composition or dosage form from, that containing a GABA analog prodrug of Formula (I) or Formula (II). In certain embodiments, compounds provided by the present disclosure may be administered prior or subsequent to administration of an additional therapeutic agent. In certain embodiments of combination therapy, the combination therapy comprises alternating between administering a composition provided by the present disclosure and a composition comprising an additional therapeutic agent, e.g., to minimize adverse side effects associated with a particular drug. When a compound provided by the present disclosure is administered concurrently with another therapeutic agent that potentially may produce adverse side effects including, but not limited to, toxicity, the therapeutic agent may advantageously be administered at a dose that falls below the threshold at which the adverse side effect is elicited.

The weight ratio of a compound provided by the present disclosure to a second therapeutic agent may be varied and may depend upon the effective dose of each agent. A therapeutically effective dose of each compound will be used. Thus, for example, when a GABA analog prodrug of Formula (I) or Formula (II) is combined with another therapeutic agent, the weight ratio of the compound provided by the present disclosure to the second therapeutic agent may be from about 1000:1 to about 1:1000, and in certain embodiments, from about 200:1 to about 1:200.

Combinations of a GABA analog prodrug of Formula (I) or Formula (II) and a second therapeutic agent may also be within the aforementioned range, but in each case, an effective dose of each active compound may be used. In such combinations a compound provided by the present disclosure and second therapeutic agent may be administered separately or in conjunction. In addition, the administration of one element may be prior to, concurrent with, or subsequent to the administration of another therapeutic agent(s). Accordingly, compounds of Formula (I) or Formula (II) may be used alone or in combination with other therapeutic agents that are known to be beneficial in treating or preventing tinnitus or other therapeutic agents that affect receptors or enzymes that either increase the efficacy, safety, convenience, or reduce unwanted side effects or toxicity of the compounds provided by the present disclosure. A prodrug of Formula (I) or Formula (II) and the other therapeutic agent may be co-administered, either in concomitant therapy or in a fixed combination. The additional therapeutic agent may be administered by the same or different route than the route used to administer a GABA analog prodrug of Formula (I) or Formula (II), or pharmaceutical composition thereof.

In certain embodiments, a GABA analog prodrug of Formula (I) or Formula (II) or a pharmaceutical composition thereof may be administered to a patient for the treatment or prevention of tinnitus in combination with a therapy or treatment known or believed to be effective in the treatment or prevention of tinnitus, or in certain embodiments, a disease, disorder, or condition associated with tinnitus. A second therapeutic agent for treating or preventing tinnitus may have one or more of analgesic, anesthetic, sodium channel blocker, antiedemic, analgesic, and antipyretic properties. Analgesics include, for example, steroidal anti-inflammatory agents, non-steroidal anti-inflammatory agents, selective COX-2 inhibitors, and narcotics. Examples of analgesics include, for example, acetaminophen, amitriptyline, aspirin, buprenorphine, celecoxib, clonidine, codeine, diclofenac, diflunisal, etodolac, fenoprofen, fentanyl, flurbiprofen, hydromorphone, hydroxyzine, ibuprofen, imipramine, indomethacin, ketoprofen, ketorolac, levorphanol, meperidine, methadone, morphine, naproxen, oxycodone, piroxicam, propoxyphene, refecoxib, sulindac, tolmetin, tramadol, valdecoxib, and combinations of any of the foregoing. In certain embodiments, a compound provided by the present disclosure or pharmaceutical composition thereof may be administered with a N-methyl-D-aspartate (NMDA) receptor antagonist that binds to the NMDA receptor at the competitive NMDA antagonist binding site, the non-competitive NMDA antagonist binding site within the ion channel, or to the glycine site. Examples of NMDA receptor antagonists include amantadine, D-2-amino-5-phosphonopentanoic acid (D-AP5), 3-((±)-2-carboxypiperazin-4-yl)-propyl-1-phosphonic acid (CCP), conantokins, 7-chlorokynurenate (7-CK), dextromethorphan, ifenprodil, ketamine, memantine, dizocilpine, gacyclidine, licostinel, phencyclidine, riluzole, traxoprodil, and combinations of any of the foregoing (see e.g., Sands, U.S. Pat. No. 5,716,961; and Guitton et al., U.S. Application Publication No. 2006/0063802). A GABA analog prodrug of Formula (I) or Formula (II), or pharmaceutical composition thereof may also be used in conjunction with non-pharmacological tinnitus therapies such as, for example, avoidance of ototoxic medications, reduced consumption of alcohol, caffeine and nicotine, reduced stress, the use of background noises or maskers, behavioral therapies such as hypnosis, cognitive therapy, biofeedback, tinnitus retraining therapy

GABA analog prodrugs of Formula (I) or Formula (II) may also be administered in conjunction with drugs that are useful for treating symptoms associated with tinnitus such as depression and anxiety. Examples of drugs useful for treating depression include, for example, alprozolam, amitriptyline, amoxapine, bupropion, citalopram, clomipramine, desipramine, doxepin, escitalopram, fluoxetine, fluvoxamine, imipramine, maprotiline, methylphenidate, mirtazapine, nefazodone, nortriptyline, paroxetine, protriptyline, sertraline, trazodone, venlafaxine, and combinations of any of the foregoing. Examples of drugs useful for treating anxiety include, for example, alprazolam, atenolol, busipirone, chlordiazepoxide, clonidine, clorazepate, diazepam, doxepin, escitalopram, halazepam, hydroxyzine, lorazepam, nadolol, oxazepam, paroxetine, prochlorperazine, trifluoperazine, venlafaxine, and combinations of any of the foregoing.

### EXAMPLES

The invention is further described by reference to the following examples, which describe synthesis of GABA analog prodrugs of Formula (I) or Formula (II), preparation of sustained release dosage forms comprising GABA analog prodrugs of Formula (I) or Formula (II) and methods of treating or preventing tinnitus comprising administering GABA analog prodrugs of Formula (I) or Formula (II). It will be apparent to those skilled in the art that many modifications, both to materials and methods, may be practiced without departing from the scope of the invention.

In the examples below, the following abbreviations have the following meanings. If an abbreviation is not defined, the generally accepted meaning applies.
cm=centimeter
g=gram
h=hour
J=Joules
kp=kilopascal
kg=kilogram
kV=kilovolt
L=liter
LC/MS=liquid chromatography/mass spectroscopy
mA=milliamps
mg=milligram
min=minute
mol=moles
mL=milliliter
mm=millimeter
μg=microgram
μL=microliter
μM=micromolar
v/v=volume to volume

### Example 1

### 1-{[(α-Isobutanoyloxyethoxy)carbonyl]aminomethyl}-1-Cyclohexane Acetic Acid via a Trimethylsilyl Ester Intermediate

### Step A: 1-{[(α-Chloroethoxy)carbonyl]aminomethyl}-1-Cyclohexane Acetic Acid

To a 5-liter, 3-neck, round bottom flask containing dichloromethane (1.6 L) was added gabapentin (120.4 g, 0.704 mol) followed by triethylamine (294 mL, 2.11 mol). Chlorotrimethylsilane (178 mL, 1.40 mol) was slowly added while maintaining the reaction temperature below 15° C. and the resulting suspension was stirred for 30 min. 1-Chloroethyl chloroformate (100 g, 0.704 mol) was then added slowly while maintaining the temperature below 15° C. After the addition was complete, additional triethylamine (88 mL, 0.63 mol) was added and the resulting suspension was stirred at room temperature for 30 min. The resulting silyl ester was converted via acidic work-up to the corresponding acid by washing the reaction mixture with water (2×1 L), followed by 1N HCl (2×2 L) then brine (2×500 mL). After drying over anhydrous sodium sulfate and removal of the solvent in vacuo, the crude product (190 g) was obtained as an orange oil and used in Step B without further purification. ¹H NMR (CDCl₃, 400 MHz): δ 1.41-1.57 (m, 10H), 1.78 (d, 3H), 2.33 (s, 2H), 3.27 (d, 2H), 5.42 (br. s, 1H), 6.55 (q, 1H).

### Step B: 1-{[(α-Isobutanoyloxyethoxy)carbonyl]aminomethyl}-1-Cyclohexane Acetic Acid (3)

To a 3-liter, 3-neck, round bottom flask was added isobutyric acid (254 g, 2.9 mol) followed by triethylamine (395 mL, 2.84 mol). The reaction mixture was cooled to room temperature and a solution of crude acid from the above reaction step (190 g, 0.69 mol) in dichloromethane (80 mL) was added in a controlled fashion while maintaining the temperature below 30° C. The resulting pale yellow solution was stirred overnight. The reaction mixture was then diluted with one volume of dichloromethane and washed with water (6×500 mL), aqueous potassium bicarbonate (3×500 mL), and brine (2×500 mL). After drying over anhydrous sodium sulfate, removal of the solvent in vacuo afforded the crude product as a dark red oil (87 g). A portion (35 g) of this product was loaded onto an 800 g Biotage™ normal phase silica gel flash column and eluted with 40% diethyl ether in hexane (6 L), which after removal of the solvent in vacuo afforded the product as a colorless oil (13.5 g). This was repeated with a second 35 g portion of crude product yielding a further 13.5 g of 1-{[(α-isobutanoyloxyethoxy)carbonyl]aminomethyl}-1-cyclohexane acetic acid. A sample of the product (25 g) was recrystallized by dissolution in heptane (325 mL) at 70° C., followed by slow cooling to room temperature. The white crystalline product (23 g) was isolated by filtration. Melting point: 63-64° C.

### Example 2

### 1-{[(α-Isobutanoyloxyethoxy)carbonyl]aminomethyl}-1-Cyclohexane Acetic Acid via an Allyl Ester Intermediate

### Step A: Allyl 1-Aminomethyl-1-Cyclohexane Acetate Hydrochloride

A dry 3 L, three-neck, round-bottomed flash fitted with a magnetic stirring bar and a 500 mL pressure-equalizing addition funnel was flushed with nitrogen gas. The flask was charged with gabapentin (171 g, 1.0 mol) and allyl alcohol (1 L, 852 g, 14.6 mol) and the entire mixture was cooled to 0° C. in an ice-water bath. Thionyl chloride (225 mL, 360 g, 3.0 mol) was added dropwise over a period of 1 h to the stirred solution. The reaction mixture was allowed to stir at room temperature for 16 h, then was diluted with ethyl ether (2 L) and cooled to 0° C. while stirring. After several minutes white crystals formed, which were collected by filtration. The crude product was recrystallized from a 1/3 (v/v) mixture of ethanol and ethyl ether (2 L) to give the product as a white solid (220 g, 88%). m.p.: 138-142° C. ¹H NMR (CD₃OD, 400 MHz): δ 1.36-1.54 (m, 10H), 2.57 (s, 2H), 3.05 (s, 2H), 4.61 (d, J=6 Hz, 2H), 5.22 (dd, J=10.4, 1.2 Hz, 1H), 5.33 (dd, J=17.2, 1.4 Hz, 1H), 5.90-6.00 (m, 1H). MS (ESI) m/z 212.0 (M+Cl)⁺.

### Step B: Allyl 1-{[(α-Chloroethoxy)carbonyl]aminomethyl}-1-Cyclohexane Acetate

To a solution of the above hydrochloride salt (220 g, 0.89 mol) in dichloromethane (1 L) was slowly added 1-chloroethyl chloroformate (101.7 mL, 132.3 g, 0.92 mol). The reaction mixture was cooled to 0° C. and 4-methylmorpholine (205 mL, 188.9 g, 1.87 mol) slowly added over a period of 1 h while maintaining a temperature of less than 10° C. The resulting turbid solution was stirred at room temperature for 1 h. Ethanol (150 mL) was added and the reaction mixture was stirred at room temperature for 1 h. The reaction mixture was then diluted with ether (2.5 L), washed with water (1 L) and brine (1 L). The organic phase was dried over sodium sulfate and concentrated to give the title compound as a light yellow viscous liquid (282 g, 100%). ¹H NMR (CDCl₃, 400 MHz): δ 1.35-1.58 (m, 10H), 1.78 (d, J=5.6 Hz, 3H), 2.32 (s, 2H), 3.22 (d, J=6.8 Hz, 2H), 4.57 (d, J=5.6 Hz, 2H), 5.25 (dd, J=10.4, 1 Hz, 1H), 5.32 (dd, J=17.2, 1.6 Hz, 1H), 5.52 (br, 1H, NH), 5.90-5.94 (m, 1H), 6.54 (q, J=5.6 Hz, 1H).

### Step C: Allyl 1-{[(α-Isobutanoyloxyethoxy)carbonyl]aminomethyl}-1-Cyclohexane Acetate

To a mixture of isobutyric acid (432 mL, 391.5 g, 4.4 mol) and 4-methylmorpholine (488 mL, 449 g, 4.4 mol) was added a solution of the chlorocarbamate from the previous step (282 g, 0.88 mol) in isobutyric acid (432 mL, 391.5 g, 4.4 mol). The addition occurred at 0° C. over a period of 30 min. The resulting turbid solution was stirred at room temperature for 16 h. The reaction mixture was diluted with ether (2.5 L) and washed with water (3×500 mL) followed by 10% aqueous potassium bicarbonate (6×500 mL) then brine (500 mL). The organic phase was dried over sodium sulfate and concentrated to yield the title compound as a viscous liquid (328 g, 100%). ¹H NMR (CDCl₃, 400 MHz): δ 1.15 (d, J=7.2 Hz, 6H), 1.35-1.58 (m, 10H), 2.31 (s, 2H), 2.51 (m, 1H), 3.19 (d, J=5.6 Hz, 2H), 4.56 (d, J=5.6 Hz, 2H), 5.24 (dd, J=10, 1 Hz, 1H), 5.32 (dd, J=17, 1.2 Hz, 1H), 5.35 (br, 1H), 5.84-5.94 (m, 1H), 6.78 (q, J=5.6 Hz, 1H). MS (ESI) m/z 392.24 (M+H)⁺.

### Step D: Deprotection of Allyl 1-{[(α-Isobutanoyloxyethoxy)carbonyl]-aminomethyl}-1-Cyclohexane Acetate

To a stirred suspension of ammonium formate (112 g, 1.7 mol) in ethanol (500 mL) was added the above allyl ester (328 g, 0.88 mol) together with 10% Pd/C (15 g) under a nitrogen atmosphere. After 6 h, the reaction mixture was worked-up by filtering off the catalyst. The catalyst was washed with ethanol (2×250 mL) and the filtrates were combined and evaporated. The crude product was dissolved in ether (2 L) and the organic phase was washed with 2N HCl (2×2 L) to convert the ammonium salt into the acid form, followed by washing with water (1 L) and brine (1 L). The ether layer was dried over sodium sulfate and concentrated to give the crude product as a viscous liquid (240 g, 82%).

### Step E: Crystallization of 1-{[(α-Isobutanoyloxyethoxy)carbonyl]-aminomethyl}-1-Cyclohexane Acetic Acid

A 3 L round-bottom flask was equipped with a heating oil bath, a nitrogen inlet adapter, an internal thermometer, an overhead mechanical stirrer, and a reflux condenser. The flask was flushed with nitrogen and charged with a 1/10 (v/v) mixture of ethyl acetate/heptane (1.2 L) and the crude product from the preceding reaction (240 g). The flask was heated until the product dissolved, then cooled according to the following schedule:

| | Time | Internal | | |
|---|---|---|---|---|
| Entry | (min) | Temp. (° C.) | Appearance | Remarks |
| 1 | 0 | 18 | Solid in solvent | Started heating oil bath |
| 2 | 10 | 48 | Turbid | Slow dissolution of |
| | | | | product |
| 4 | 20 | 58 | Clear solution | Turn off oil bath |
| 5 | 25 | 60 | Clear solution | Maximum temp. reached |
| 6 | 45 | 43 | Turbid | Compound crystallizing |
| 7 | 60 | 36 | Milky solution | Seeded with pure ref. |
| | | | | material |
| 8 | 90 | 24 | Solid in solution | — |

The flask was then cooled to 4° C. overnight with stirring (cooling improves the yield). The product was filtered and washed with heptane (2×100 mL), then dried under reduced pressure (25 mm of Hg (0.033 atm)) at 30° C. for 18 h to yield 1-{[(α-isobutanoyloxyethoxy)carbonyl]aminomethyl}-1-cyclohexane acetic acid (185 g) as a white crystalline solid.

### Example 3

### X-Ray Powder Diffraction Analysis of Crystalline

### 1-{[(α-Isobutanoyloxyethoxy)carbonyl]aminomethyl}-1-Cyclohexane Acetic Acid

X-ray powder diffractograms (XRPDs) of crystalline samples of 1-{[(α-isobutanoyloxyethoxy)carbonyl]aminomethyl}-1-cyclohexane acetic acid produced according to Examples 1 and 2 above were measured with a Bruker D8 Discover X-ray powder diffractometer using Cu Kα radiation. The instrument is equipped with parallel beam optics and a two-dimensional HI-STAR area detector. The tube voltage and amperage were set to 40 kV and 40 mA, respectively. The collimated X-ray beam was reduced to a spot size of about 0.5 mm in diameter. The area detector was placed 15 cm from the center of the goniometer and the angular resolution is approximately 0.033°/pixel. The detector covered a range of 35° in 2-theta (2θ) within one frame. The angle between the X-ray beam and the horizontal sample plate was set to 4° and the center of the area detector was set to an angle of 18°. This geometry allowed the measurement of 2-theta from 4.5° to 39.5° within one frame. The typical averaging time was 3 minutes for each XRPD pattern collected. A corundum sample (NIST 1976) was used to calibrate the XRPD instrument. Both samples gave equivalent diffractogram patterns.

### Example 4

### Melting Point and Differential Scanning Calorimetry Analysis of Crystalline 1-{[(α-Isobutanoyloxyethoxy)carbonyl]aminomethyl}-1-Cyclohexane Acetic Acid

Melting points of crystalline samples of 1-{[(α-isobutanoyloxyethoxy)carbonyl]aminomethyl}-1-cyclohexane acetic acid produced according to Examples 1 and 2 above were measured using an Electrothermal 9200 melting point apparatus and determined to be 63-64° C.

Differential scanning calorimetry (DSC) analysis of crystalline samples of 1-{[(α-isobutanoyloxyethoxy)carbonyl]aminomethyl}-1-cyclohexane acetic acid produced according to Examples 1 and 2 above were measured using a Perkin Elmer Series 7 instrument, scanning from 25° C. to 250° C. at a scan rate of 5° C./min. A test portion of the sample was placed in an aluminum pan and the cap crimped to eliminate any visible seam between the cap and the pan. An empty pan was prepared in the same manner as a blank. The pans were placed in the differential scanning calorimeter. The material was run at the appropriate temperature program (Equilibration at Initial Temp, Isothermal, Ramp Rate, Final Temp). DSC analysis showed an endothermic transition with an onset temperature of 58.3° C. and a ΔH of 72.39 J/g. At the peak endotherm of 63-64° C. the sample visibly melted.

### Example 5

### {[(1-Isobutanoyloxyethoxy)carbonyl]aminomethyl}-1-Cyclohexane Acetic Acid (3)

To a solution of gabapentin (6.8 g, 0.04 mol) in water (40 mL) was added a solution of [(1-isobutanoyloxyethoxy)carbonyloxy]succinimide (10 g, 0.036 mol) in acetonitrile (40 mL) over a period of 30 min. The reaction was stirred at ambient temperature for 3 hours. The reaction mixture was diluted with methyl tert-butyl ether (200 mL), washed with water (2×100 mL) and brine (50 mL). The organic phase was separated, dried over anhydrous sodium sulfate, filtered, and concentrated in vacuo to afford the title compound as a white solid (12 g, quantitative).

The following procedure was used to crystallize the title compound. The solid compound (12 g) was suspended in methylcyclohexane: methyl tert-butyl ether 10:1 (60 mL). The suspension was slowly heated up to 50° C. over a period of 30 min. The clear solution was then allowed to cool to room temperature. The turbid mixture was seeded with 5 mg of the title compound in crystalline form. The mixture was further cooled to 0-4° C. for 2 h. The solid product was filtered and washed with methylcyclohexane (2×10 mL) to yield the title compound (3) as a white crystalline solid (10 g, 83% yield). The crystalline solid material had a melting point of about 64-66° C. as measured by open capillary melting point determination.

### Example 6

### Preparation of a Sustained Release Oral Dosage Form of 1-{[(α-Isobutanoyloxyethoxy)carbonyl]aminomethyl}-1-Cyclohexane Acetic Acid (3)

Sustained release oral dosage forms containing the gabapentin prodrug, 1-{[(α-isobutanoyloxyethoxy)carbonyl]aminomethyl}-1-cyclohexane acetic acid (compound (3)), was prepared according the procedure disclosed in Cundy, U.S. Application Publication No. 2006/0141034, which is incorporated by reference herein in its entirety. Oral sustained release dosage form tablets containing compound (3) were made having the ingredients shown in Table 1:

TABLE 1

| | | Amount/Tablet | Composition | Ingredient |
|---|---|---|---|---|
| Ingredient | Manufacturer | (mg/tablet) | (wt %) | Category |
| Compound (3) | XenoPort | 600.00 | 45.80 | Prodrug |
| | (Santa Clara, | | | |
| | CA) | | | |
| Dibasic Calcium | Rhodia | 518.26 | 39.56 | Diluent |
| Phosphate, USP | (Chicago, IL) | | | |
| Glyceryl | Gattefosse | 60.05 | 4.58 | Lubricant/ |
| Behenate, NF | (Saint Pirest, | | | Release |
| | Cedex, France) | | | controlling |
| | | | | agent |
| Talc, USP | Barrett Minerals | 80.02 | 6.11 | Anti-adherent |
| | (Mount Vernon, | | | |
| | IN) | | | |
| Colloidal Silicon | Cabot | 5.43 | 0.41 | Glidant |
| Dioxide, NF | (Tuscola, IL) | | | |
| Sodium Lauryl | Fisher | 24.00 | 1.84 | Surfactant |
| Sulfate, NF | (Fairlawn, NJ) | | | |
| Magnesium | Mallinckrodt | 22.22 | 1.69 | Lubricant |
| Stearate, NF | (Phillipsburg, | | | |
| | NJ) | | | |
| Total | | 1310.00 | 100 | |

The tablets were made according to the following steps. Compound (3), dibasic calcium phosphate, glyceryl behenate, talc, and colloidal silicon dioxide were weighed out, screened through a #20 mesh screen and mixed in a V-blender for 15 minutes. The slugging portion of the sodium lauryl sulfate was weighed and passed through a #30 mesh screen. The slugging portion of the magnesium stearate was weighed and passed through a #40 mesh screen. Screened sodium lauryl sulfate and magnesium stearate were added to the V-blender and blended for 5 min. The blend was discharged and compressed into slugs of approximately 400 mg weight on a tablet compression machine. The slugs were then passed through a Comil 194 Ultra mill (Quadro Engineering, Inc., Millburn, N.J.) to obtain the milled material for further compression. The tableting portion of the sodium lauryl sulfate was weighed and passed through a #30 mesh screen. The tableting portion of the magnesium stearate was weighed and passed through a #40 mesh screen. The milled material and the tableting portions of the sodium lauryl sulfate and magnesium stearate were added to the V-blender and blended for 3 min. The blended material was discharged and compressed to form tablets having a total weight of 1310 mg and a compound (3) loading of 600 mg (45.8 wt %). The tablets had a mean final hardness of 16.1 to 22.2 kPa (158 to 218 N).

### Example 7

### Pharmacokinetics of Orally Administered 1-{[(α-Isobutanoyloxyethoxy)carbonyl]aminomethyl}-1-Cyclohexane Acetic Acid (3)

A randomized, crossover, fed/fasted single-dose study of the safety, tolerability, and pharmacokinetics of oral administration of 1-{[(α-isobutanoyloxyethoxy)carbonyl]aminomethyl}-1-cyclohexane acetic acid (3) in healthy adult subjects was conducted. The oral sustained release dosage form of Example 6 was used in this study. The study was designed to evaluate the performance of this formulation in humans in comparison with the commercial gabapentin capsule formulation (Neurontin®, Pfizer). Twelve healthy adult volunteers (7 males and 5 females) participated in the study. Mean body weight was 75.6 kg. All subjects received two different treatments in a random order with a one-week washout between treatments. The two treatments were: (A) a single oral dose of Example 6 tablets (2×600 mg) after an overnight fast; and (B) a single oral dose of Example 6 tablets (2×600 mg) after a high fat breakfast.

Blood and plasma samples were collected from all subjects prior to dosing, and at 0.5, 1, 1.5, 2, 3, 4, 6, 8, 12, 18, 24, and 36 hours after dosing. Urine samples were collected from all subjects prior to dosing, and complete urine output was obtained at the 0-4 h, 4-8 h, 8-12 h, 12-18 h, 18-24 h, and 24-36 h intervals after dosing. Blood samples were quenched immediately with methanol and stored frozen at <70° C. Sample aliquots were prepared for analysis of gabapentin and compound (3) using sensitive and specific LC/MS/MS methods.

The mean ±SD Cₘₐₓ for gabapentin in blood after oral dosing of the tablets (fasted) was 4.21±1.15 μg/mL. Following administration of the tablets after a high fat breakfast, the Cₘₐₓ of gabapentin in blood was further increased to 6.24±1.55 μg/mL. The mean ±SD AUC for gabapentin in blood after oral dosing of the tablets (fasted) was 54.5±12.2 μg·h/mL. Following administration of the tablets after a high fat breakfast, the AUC of gabapentin in blood was further increased to 83.0±21.8 μg·h/mL. In the presence of food, exposure to gabapentin after oral administration of the tablets increased an additional 52% compared to that in fasted subjects.

The time to peak blood levels (Tₘₐₓ) of gabapentin was significantly delayed after oral administration of the tablets. In fasted subjects, oral administration of the tablets gave a gabapentin Tₘₐₓ of 5.08±1.62 h. This compares to a typical Tₘₐₓ of immediate release gabapentin of about 2-4 h. The gabapentin Tₘₐₓ after oral administration of the tablets was further delayed to 8.40±2.07 h in the presence of food. The apparent terminal elimination half-life for gabapentin in blood was similar for all treatments: 6.47±0.77 h for the tablets in fasted subjects, and 5.38±0.80 h for the tablets in fed subjects.

Following oral administration of the tablets, the percent of the gabapentin dose recovered in urine was 46.5±15.8% for fasted subjects and 73.7±7.2% for fed subjects.

Exposure to intact prodrug in blood after oral administration of the tablets was low. After oral dosing of the tablets in fasted subjects, concentrations of intact compound (3) in blood reached a maximum of 0.040 μg/mL, approximately 1.0% of the corresponding peak gabapentin concentration. Similarly, the AUC of compound (3) in blood of these subjects was 0.3% of the corresponding AUC of gabapentin in blood. After oral dosing of the tablets in fed subjects, concentrations of intact compound (3) in blood reached a maximum of 0.018 μg/mL, approximately 0.3% of the corresponding peak gabapentin concentration. Similarly, the AUC of compound (3) in blood of these subjects was less than 0.1% of the corresponding AUC of gabapentin in blood.

### Example 8

### Animal Model for Assessing Therapeutic Efficacy of Prodrugs for Treating Tinnitus

The efficacy of a GABA analog prodrug of Formula (I) or Formula (II) maybe assessed using animal models of tinnitus in which unilateral noise trauma is used to induce tinnitus (see e.g., Bauer and Brozoski, J Assoc Res Otolarynology 2001, 2(1), 54-64; and see also Guitton et al., U.S. Application Publication No. 2006/0063802.

Long-Evans rats are first behaviorally acclimated to lever-press for food pellets and then conditioned to respond to a distinctive and standard way to auditory test stimuli. After conditioning, the animals are separated into groups and exposed to unilateral noise trauma for 0, 1, or 2 hours. Animals are anesthetized, placed in a stereotaxic head frame, and unilaterally exposed once to narrowband noise with a peak intensity of 105 dB centered at 16 kHz for 0, 1, or 2 hours before or after behavioral training and testing. The animals are then administered a GABA analog prodrug of Formula (I) or Formula (II) and suppression of the conditioned response determined and compared to a control group not exposed to noise trauma.

### Example 9

### Method for Assessing Therapeutic Efficacy of Prodrugs for Treating Tinnitus in Humans

The efficacy of GABA analog prodrugs of Formula (I) or Formula (II) maybe assessed using, for example, the method described by Bauer and Brozoski, Laryngoscope 2006, 116, 675-681 or by Folmer, BMC Ear, Nose and Throat Disorders 2002, 2(3), 1-9.

Subjects are screened using pre-established inclusion and exclusion criteria and selected for their ability to perform a psychophysical loudness matching task using pure tones and broad-band noise (BBN). Examples of inclusion criteria include, for example, age, type of tinnitus, e.g., continuous or pulsed, duration of tinnitus, Tinnitus Handicap Questionnaire (THQ) score >30, Beck Depression Index (BDI)<13, and criterion performance on loudness matching task using a 1 KHz standard.

Following screening, selection and enrollment, tinnitus is evaluated before and after a GABA analog prodrug of Formula (I) or Formula (II) is administered to a subject. Hearing thresholds are evaluated using an objective stimulus loudness match and a tinnitus loudness matching procedure.

Prior to enrollment, subjects are screened for proficiency in a psychophysical matching task. In the objective stimulus loudness matching procedure, subjects match a binaural 1 KHz standard tone at 20 dB sensation levels to each of five binaural comparison stimuli (BBN, 0.5, 1, 2, and 4 KHz). The loudness match is obtained using a forced two-choice procedure. Each trial begins with the simultaneous presentation of a visual cue and the 1 KHz standard followed by the presentation of the second visual cue and the comparison stimulus. Subjects are instructed to indicate whether the standard and comparison stimulus sounded the “same” or “different” in loudness by clicking an on-screen button. An ascending-descending method of limits procedure is used. Subjects are screened using this loudness-matching test and are required to meet inclusion criteria of efficiency (completion time ≦1 h) and reliability (standard deviation of match levels ≦5 dB).

The tinnitus loudness matching procedure differs from the objective stimulus loudness matching procedure in that the initial presentation on each trial is a null presentation during which an on-screen message instructs subjects to listen closely to their tinnitus. During this initial cue (1 sec) subjects are instructed to use their perception of tinnitus as the standard stimulus. Subjects are instructed to click a “same loudness” button when the loudness of the comparison stimulus matches the loudness of their tinnitus. The presentation order of the comparison stimuli (BBN, 0.5, 1, 2, and 4 KHz) is randomized, and each ascending and descending stimulus series is repeated once, for a total of four tinnitus loudness matches at each of the five comparison stimuli. The intensities of the loudness-match points are recorded and converted to sensation levels of tinnitus loudness using the hearing threshold determined in each session for the comparison stimuli. Psychoacoustically determined tinnitus loudness is reported as dB HL of the maximum sensation-level match obtained within a session.

Assessment sessions are performed at the initiation of the study and at intervals during the study. Subjects may be given placebo only, test compound only, a variable including escalating or deescalating dose of a GABA analog prodrug of Formula (I) or Formula (II), or a combination of placebo and test compound during the course of a study. The duration of the study may be for a few hours, days, weeks, months, or years.

Primary outcome measures are psychoacoustically determined tinnitus loudness and perceived tinnitus handicap. Tinnitus handicap was determined using the Tinnitus Handicap Questionnaire, which provides a global score and subscores related to emotional, functional, and cognitive aspects of tinnitus (see e.g., Kuk et al., Ear Hear 1990, 11, 434-45). Secondary outcome measures include general health and quality of life factors determined, for example, using the General Health Survey Short form (RAND 36-Item Health Survey, 1.0, Rand Health, Santa Monica, Calif.) and the Tinnitus Experience Questionnaire, a set of seven scaled questions that evaluate the experiential sensory features of tinnitus. Other questionnaires for assessing tinnitus may be used such as those described in Soderman et al., Otol Neurotol 2001, 22(4), 526-33; Henry et al., Am J Audiol 2005, 14(1), 21-48; and others known to those skilled in the art.

Finally, it should be noted that there are alternative ways of implementing the embodiments disclosed herein. Accordingly, the present embodiments are to be considered as illustrative and not restrictive, and the claims are not to be limited to the details given herein, but may be modified within the scope and equivalents thereof described herein.

What is claimed is:

1. A method of treating tinnitus in a patient, comprising administering to a patient in need of such treatment a therapeutically effective amount of at least one compound chosen from Formula (I) and Formula (II): 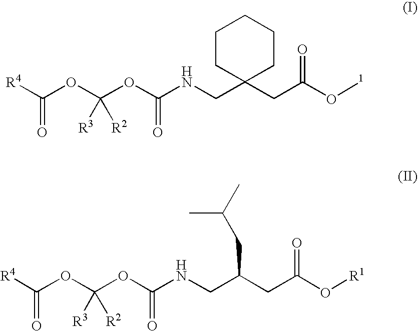
a pharmaceutically acceptable salt thereof, a pharmaceutically acceptable solvate of any of the foregoing, and a pharmaceutically acceptable N-oxide of any of the foregoing, wherein:
R¹ is chosen from hydrogen, alkyl, substituted alkyl, aryl, substituted aryl, arylalkyl, substituted arylalkyl, cycloalkyl, substituted cycloalkyl, cycloheteroalkyl, substituted cycloheteroalkyl, heteroalkyl, substituted heteroalkyl, heteroaryl, substituted heteroaryl, heteroarylalkyl, and substituted heteroarylalkyl;
R² and R³ are independently chosen from hydrogen, alkyl, substituted alkyl, alkoxycarbonyl, substituted alkoxycarbonyl, aryl, substituted aryl, arylalkyl, substituted arylalkyl, carbamoyl, substituted carbamoyl, cycloalkyl, substituted cycloalkyl, heteroalkyl, substituted heteroalkyl, cycloheteroalkyl, substituted cycloheteroalkyl, heteroaryl, substituted heteroaryl, heteroarylalkyl, and substituted heteroarylalkyl, or R² and R³ together with the carbon atom to which they are bonded form a cycloalkyl, substituted cycloalkyl, cycloheteroalkyl, or substituted cycloheteroalkyl ring; and
R⁴ is chosen from acyl, substituted acyl, alkyl, substituted alkyl, aryl, substituted aryl, arylalkyl, substituted arylalkyl, cycloalkyl, substituted cycloalkyl, cycloheteroalkyl, substituted cycloheteroalkyl, heteroalkyl, substituted heteroalkyl, heteroaryl, substituted heteroaryl, heteroarylalkyl, and substituted heteroarylalkyl.

2. The method of claim 1, wherein R¹ is hydrogen.

3. The method of claim 1, wherein at least one of R² and R³ is other than hydrogen.

4. The method of claim 1, wherein R² and R³ are independently chosen from hydrogen and C₁₋₆ alkyl.

5. The method of claim 1, wherein R³ is chosen from methyl, ethyl, n-propyl, isopropyl, n-butyl, isobutyl, and sec-butyl, and R² is hydrogen.

6. The method of claim 1, wherein R⁴ is chosen from C₁₋₆ alkyl and C₁₋₆ substituted alkyl.

7. The method of claim 1, wherein R⁴ is chosen from methyl, ethyl, n-propyl, isopropyl, n-butyl, isobutyl, sec-butyl, n-pentyl, isopentyl, sec-pentyl, neopentyl, and 1,1-diethoxyethyl.

8. The method of claim 1, wherein R¹ and R² are each hydrogen, R³ is C₁₋₆ alkyl, and R⁴ is chosen from C₁₋₆ alkyl and C₁₋₆ substituted alkyl.

9. The method of claim 1, wherein R¹ and R² are each hydrogen, R³ is chosen from methyl, ethyl, n-propyl, isopropyl, n-butyl, isobutyl, and sec-butyl, and R⁴ is chosen from methyl, ethyl, n-propyl, isopropyl, n-butyl, isobutyl, sec-butyl, n-pentyl, isopentyl, sec-pentyl, neopentyl, and 1,1-diethoxyethyl.

10. The method of any one of claims 1, 6, and 8, wherein each substituent is independently chosen from halogen, —NH₂, —OH, —CF₃, —CN, —COOH, —C(O)NH₂, —C(O)OR⁵, and —NR⁵₃⁺ wherein each R⁵ is independently C₁₋₃ alkyl.

11. The method of claim 1, wherein the compound is a compound of Formula (I) chosen from:
1-{[(α-Acetoxyethoxy)carbonyl]aminomethyl}-1-cyclohexane acetic acid;
1-{[(α-Propanoyloxyethoxy)carbonyl]aminomethyl}-1-cyclohexane acetic acid;
1-{[(α-Butanoyloxyethoxy)carbonyl]aminomethyl}-1-cyclohexane acetic acid;
1-{[(α-Isobutanoyloxyethoxy)carbonyl]aminomethyl}-1-cyclohexane acetic acid;
1-{[(α-Pivaloxyethoxy)carbonyl]aminomethyl}-1-cyclohexane acetic acid;
1-{[(α-Acetoxymethoxy)carbonyl]aminomethyl}-1-cyclohexane acetic acid;
1-{[(α-Propanoyloxymethoxy)carbonyl]aminomethyl}-1-cyclohexane acetic acid;
1-{[(α-Butanoyloxymethoxy)carbonyl]aminomethyl}-1-cyclohexane acetic acid;
1-{[(α-Isobutanoyloxymethoxy)carbonyl]aminomethyl}-1-cyclohexane acetic acid;
1-{[(α-Pivaloxymethoxy)carbonyl]aminomethyl}-1-cyclohexane acetic acid;
1-{[(α-Acetoxypropoxy)carbonyl]aminomethyl}-1-cyclohexane acetic acid;
1-{[(α-Propanoyloxypropoxy)carbonyl]aminomethyl}-1-cyclohexane acetic acid;
1-{[(α-Butanoyloxypropoxy)carbonyl]aminomethyl}-1-cyclohexane acetic acid;
1-{[(α-Isobutanoyloxypropoxy)carbonyl]aminomethyl}-1-cyclohexane acetic acid;
1-{[(α-Pivaloxypropoxy)carbonyl]aminomethyl}-1-cyclohexane acetic acid;
1-{[(α-Acetoxyisopropoxy)carbonyl]aminomethyl}-1-cyclohexane acetic acid;
1-{[(α-Propanoyloxyisopropoxy)carbonyl]aminomethyl}-1-cyclohexane acetic acid;
1-{[(α-Butanoyloxyisopropoxy)carbonyl]aminomethyl}-1-cyclohexane acetic acid;
1-{[(α-Isobutanoyloxyisopropoxy)carbonyl]aminomethyl}-1-cyclohexane acetic acid;
1-{[(α-Pivaloxyisopropoxy)carbonyl]aminomethyl}-1-cyclohexane acetic acid;
1-{[(α-Acetoxybutoxy)carbonyl]aminomethyl}-1-cyclohexane acetic acid;
1-{[(α-Propanoyloxybutoxy)carbonyl]aminomethyl}-1-cyclohexane acetic acid;
1-{[(α-Butanoyloxybutoxy)carbonyl]aminomethyl}-1-cyclohexane acetic acid;
1-{[(α-Isobutanoyloxybutoxy)carbonyl]aminomethyl}-1-cyclohexane acetic acid; and
1-{[(α-Pivaloxybutoxy)carbonyl]aminomethyl}-1-cyclohexane acetic acid;
a pharmaceutically acceptable salt of any of the foregoing, a pharmaceutically acceptable solvate of any of the foregoing, and a pharmaceutically acceptable N-oxide of any of the foregoing.

12. The method of claim 1, wherein the compound is a compound of Formula (I) and is 1-{[(α-isobutanoyloxyethoxy)carbonyl]aminomethyl}-1-cyclohexane acetic acid or a pharmaceutically acceptable salt thereof, a pharmaceutical acceptable solvate of any of the foregoing, or a pharmaceutically acceptable N-oxide of any of the foregoing.

13. The method of claim 12, wherein the 1-{[α-isobutanoyloxyethoxy)carbonyl]aminomethyl}-1-cyclohexane acetic acid is crystalline.

14. The method of claim 13, wherein the crystalline 1-{[α-isobutanoyloxyethoxy)carbonyl]aminomethyl}-1-cyclohexane acetic acid has characteristic absorption peaks at 7.0°±0.3°, 8.2°±0.3°, 10.5°±0.3°, 12.8°±0.3°, 14.9°±0.3°, 16.4°±0.3°, 17.9°±0.3°, 18.1°±0.3°, 18.9°±0.3°, 20.9°±0.3°, 23.3°±0.3°, 25.3°±0.3°, and 26.6°±0.3° in an X-ray powder diffractogram.

15. The method of claim 13, wherein the crystalline 1-{[α-isobutanoyloxyethoxy)carbonyl]aminomethyl}-1-cyclohexane acetic acid has a melting point range from about 63° C. to about 66° C.

16. The method of claim 1, wherein the compound is a compound of Formula (II) chosen from:
3-{[(α-Acetoxyethoxy)carbonyl]aminomethyl}-5-methyl hexanoic acid;
3-{[(α-Propanoyloxyethoxy)carbonyl]aminomethyl}-5-methyl hexanoic acid;
3-{[(α-Butanoyloxyethoxy)carbonyl]aminomethyl}-5-methyl hexanoic acid;
3-{[(α-Isobutanoyloxyethoxy)carbonyl]aminomethyl}-5-methyl hexanoic acid;
3-{[(α-Pivaloxyethoxy)carbonyl]aminomethyl}-5-methyl hexanoic acid;
3-{[(α-Acetoxymethoxy)carbonyl]aminomethyl}-5-methyl hexanoic acid;
3-{[(α-Propanoyloxymethoxy)carbonyl]aminomethyl}-5-methyl hexanoic acid;
3-{[(α-Butanoyloxymethoxy)carbonyl]aminomethyl}-5-methyl hexanoic acid;
3-{[(α-Isobutanoyloxymethoxy)carbonyl]aminomethyl}-5-methyl hexanoic acid;
3-{[(α-Pivaloxymethoxy)carbonyl]aminomethyl}-5-methyl hexanoic acid;
3-{[(α-Acetoxypropoxy)carbonyl]aminomethyl}-5-methyl hexanoic acid;
3-{[(α-Propanoyloxypropoxy)carbonyl]aminomethyl}-5-methyl hexanoic acid;
3-{[(α-Butanoyloxypropoxy)carbonyl]aminomethyl}-5-methyl hexanoic acid;
3-{[(α-Isobutanoyloxypropoxy)carbonyl]aminomethyl}-5-methyl hexanoic acid;
3-{[(α-Pivaloxypropoxy)carbonyl]aminomethyl}-5-methyl hexanoic acid;
3-{[(α-Acetoxyisopropoxy)carbonyl]aminomethyl}-5-methyl hexanoic acid;
3-{[(α-Propanoyloxyisopropoxy)carbonyl]aminomethyl}-5-methyl hexanoic acid;
3-{[(α-Butanoyloxyisopropoxy)carbonyl]aminomethyl}-5-methyl hexanoic acid;
3-{[(α-Isobutanoyloxyisopropoxy)carbonyl]aminomethyl}-5-methyl hexanoic acid;
3-{[(α-Pivaloxyisopropoxy)carbonyl]aminomethyl}-5-methyl hexanoic acid;
3-{[(α-Acetoxybutoxy)carbonyl]aminomethyl}-5-methyl hexanoic acid;
3-{[(α-Propanoyloxybutoxy)carbonyl]aminomethyl}-5-methyl hexanoic acid;
3-{[(α-Butanoyloxybutoxy)carbonyl]aminomethyl}-5-methyl hexanoic acid;
3-{[(α-Isobutanoyloxybutoxy)carbonyl]aminomethyl}-5-methyl hexanoic acid; and
3-{[(α-Pivaloxybutoxy)carbonyl]aminomethyl}-5-methyl hexanoic acid;
a pharmaceutically acceptable salt of any of the foregoing, a pharmaceutically acceptable solvate of any of the foregoing, and a pharmaceutically acceptable N-oxide of any of the foregoing.

17. The method of claim 1, wherein the compound is a compound of Formula (II) and is 3-{[(α-isobutanoyloxyethoxy)carbonyl]aminomethyl}-5-methyl hexanoic acid or a pharmaceutically acceptable salt thereof, a pharmaceutically acceptable solvate of any of the foregoing, or a pharmaceutically acceptable N-oxide of any of the foregoing.

18. The method of claim 1, wherein the compound is a compound of Formula (I) and is administered in an amount from about 10 mg-equivalents to about 3,600 mg-equivalents of gabapentin per day.

19. The method of claim 1, wherein the compound is a compound of Formula (II) and is administered in an amount from about 10 mg-equivalents to about 1,200 mg-equivalents of pregabalin per day.

20. The method of claim 1, wherein the compound is administered orally.

21. The method of claim 20, comprising orally administering the compound in a sustained release oral dosage form.

22. The method of claim 21, wherein a therapeutically effective amount of gabapentin or pregabalin is maintained in the plasma of the patient for a period of at least about 4 hours after administrating the compound.

23. The method of claim 21, wherein the therapeutically effective amount of gabapentin or pregabalin is maintained in the plasma of the patient for a period of at least about 8 hours after administrating the compound.

24. The method of claim 21, wherein the therapeutically effective amount of gabapentin or pregabalin is maintained in the plasma of the patient for a period of at least 12 hours after administrating the compound.
